(19) United States
(12) Patent Application Publication
(10) Pub. No.: US 2014/0271667 A1
(43) Pub. Date: Sep. 18, 2014

(54) Methods and Compositions for Neoadjuvant Therapy

(71) Applicant: The Wistar Institute of Anatomy and Biology, Philadelphia, PA (US)

(72) Inventors: Dario C. Altieri, Philadelphia, PA (US); Young Chan Chae, Drexel Hill, PA (US)

(73) Assignee: The Wistar Institute of Anatomy and Biology, Philadelphia, PA (US)

(21) Appl. No.: 14/211,394

(22) Filed: Mar. 14, 2014

Related U.S. Application Data
(60) Provisional application No. 61/787,156, filed on Mar. 15, 2013.

(51) Int. Cl. A61K 45/06; A61K 31/395; A61K 39/395; C07K 16/40; C07K 16/18

(52) U.S. Cl. CPC A61K 45/06; C07K 16/40; C07K 16/18; A61K 39/39558; A61K 31/395
USPC 424/158.1; 424/174.1; 530/389.7; 536/23.5; 514/183; 540/461

(57) ABSTRACT

A method for inhibiting tumor cell migration or metastasis of a cancer in a mammalian subject comprises one or more of the steps of administering to a subject a therapeutically effective amount of a composition comprising a molecule that: suppresses focal adhesion kinase (FAK) activity or phosphorylation; suppresses ULK1 kinase activity; suppresses activation or signaling of the mTORC1 (Ser757) pathway; activates AMPK; activates FIP200; or activates LKB1, in a cancer cell. Still another method of inhibiting tumor cell migration involves inhibiting phosphorylation of ULK1 on Ser757 in subjects with lung cancer. Suppressing activation or signaling of the mTORC1 (Ser757) pathway in subjects is in one aspect useful in treating lung cancer.

### CROSS-REFERENCE TO RELATED APPLICATIONS

This application claims the benefit of the priority of U.S. Provisional Patent Application No. 61/787,156, filed Mar. 15, 2013, which application is incorporated herein by reference in its entirety.

### STATEMENT REGARDING FEDERALLY SPONSORED RESEARCH OR DEVELOPMENT

This invention was made with government support under Grant Nos. CA140043, HL054131, CA078810, CA118005 and CA010815 awarded by the National Institutes of Health. The government has certain rights in this invention.

### BACKGROUND OF THE INVENTION

A critical problem facing physicians treating patients with a cancer diagnosis is finding a suitable treatment for removing or treating the primary cancer while simultaneously avoiding the metastatic spread of the primary cancer to secondary locations in the body. Cancer cells typically migrate from the primary site of the tumor and invade the basement membranes lining other organs, blood vessels or other tissue of the body and replicate at the new location.

A typical course of therapy for a solid tumor cancer involves using chemotherapy to reduce the size of the tumor, followed by surgery to debulk the tumor from its primary site, followed by a second round of chemotherapy conducted with the intent of killing any residual cancer cells left in the body following surgery. However, the very act of surgical debulking commonly releases a certain number of the tumor cells that escape the primary locus and migrate to other tissue or organs. The first and post-surgery courses of chemotherapeutics or radiation also can cause side effects that can impact the patient's health and immune status.

Metabolic reprogramming of tumors (1) is being increasingly recognized as an important disease driver, controlling various aspects of malignant development and progression (2). Although energetically unfavorable (3), cancer metabolism contributes to biomass expansion (4), oncogenic signaling (5), generation of biochemical defects that further the malignant phenotype (6, 7), and transformation-associated epigenetic changes (8, 9). How tumor cells exploit a bioenergetics program to regulate malignant growth is beginning to emerge (10), but the regulators of this process are still elusive, and their link to mechanisms of advanced disease, for instance metastasis (11), has not been clearly elucidated.

In this context, tumors grow in acutely unfavorable environments, constantly exposed to oxidative stimuli and chronically depleted of oxygen and nutrients (12). Stress signals generated under these conditions antagonize tumor growth via activation of tumor suppressors (13), including liver kinase B1 (LKB1)/AMP-activated kinase (AMPK) (14), inhibition of oncogenes, for instance the mammalian target of rapamycin complex-1 (mTORC1) (15), and induction of autophagy (16), a process of cellular self-digestion (17) that is often a barrier to transformation (18). Notwithstanding, nutrient-starved tumors circumvent these challenges, and manage to acquire highly energetically-demanding traits, such as invasiveness, which heralds metastatic and lethal disease (19). A network of Heat Shock Protein-90 (Hsp90) chaperones (20) that is preferentially, if not exclusively found in mitochondria of tumor cells (21) oversee the organelle protein folding environment in tumors, antagonizing cyclophilin D (CypD)-dependent permeability transition (22), and maintaining energy production via retention of the glycolytic enzyme, hexokinase II to the mitochondrial outer membrane (23).

Thus a means for inhibiting the metastatic migration of cancer cells during conventional cancer therapies remains an elusive goal.

### SUMMARY OF THE INVENTION

In one aspect, a method for inhibiting tumor migration or metastasis of a tumor cell or cancer cell comprises interrupting the metabolic reprogramming of mitochondrial bioenergetics influenced mechanisms of tumor cell invasion and metastasis, in vivo.

In one aspect, the method involves reducing or suppressing focal adhesion kinase (FAK) activity or expression, or reducing or suppressing phosphorylation of FAK in a cancer cell.

In another aspect, the method involves reducing or suppressing ULK1 kinase activity or expression in a cancer cell.

In yet another aspect, the method involves reducing or suppressing activation, expression or signaling of the mTORC1 (Ser757) pathway in a cancer cell. In one embodiment, this method is directed for use in subjects with lung cancer.

In still another aspect, the method comprises increasing or stimulating activation or expression of AMPK or LKB1 or FIP200 in a cancer cell. In one aspect, the method involves adjusting the ratio of AMPK to mTORC1 in favor of AMPK activation.

In still another aspect, the method comprises reducing or suppressing the activation or expression of, or silencing, TRAP1 in a cancer cell.

In another aspect, the method involves administering to a subject a therapeutically effective dose of a composition comprising one or more of a molecule that reduces or suppresses focal adhesion kinase (FAK) activity or expression or phosphorylation of FAK in a cancer cell; a molecule that reduces or suppresses ULK1 kinase activity or expression; a molecule that reduces or suppresses activation, expression or signaling of the mTORC1 (Ser757) pathway; a molecule that increases or stimulates activation of AMPK or LKB1 or FIP200; or a molecule that reduces or suppresses the activation or expression of, or silences, TRAP1.

In yet another aspect, methods useful in accomplishing this tumor cell migration/metastasis inhibition include one or a combination of two or more of the above-noted method administration steps.

In one embodiment, any of the methods above involve administration of the composition prior to any cancer treatment. In another embodiment, the method occurs at a selected time during the course of the cancer therapy. In one embodiment, the dose is one or more suboptimal or non-cytotoxic doses.

In another aspect, a method comprises administering to a subject in need thereof one or more suboptimal or non-cytotoxic doses of Gamitrinib during cancer therapy. In one embodiment, these doses do not cause systemic immune suppression in the treated subject. In another embodiment, these doses reduce the metastatic migration of the cancer cells.

In yet another aspect, a composition for accomplishing one or more of the methods described here is provided.

In another aspect, use of the above-noted compositions and methods is provided as neoadjuvant therapy in the treatment of a subject with cancer.

In yet another aspect, a method of suppressing FAK kinase activity or inhibiting phosphorylation of FAK in tumor cells comprises administering to a subject in need thereof one or more suboptimal doses of Gamitrinib, wherein the doses do not cause systemic immune suppression in the subject.

Other aspects and advantages of these compositions and methods are described further in the following detailed description of the preferred embodiments thereof.

### BRIEF DESCRIPTION OF THE DRAWINGS

FIG. 1A through 1H show Gamitrinib inhibition of tumor cell motility. (A, B) Gamitrinib (Gam, 5 μM)-treated PC3 cells were analyzed for cell migration (A) or invasion (B). Mean±SEM (n=3), ***, p<0.0001. (C, D) 3-D organotypic LN229 spheroids treated with vehicle or Gamitrinib (Gam, 1.25-2.5 μM) were analyzed by phase contrast microscopy (C, top) and mask-inverted images (C, bottom) were used to quantify the length and area between the core and the invasive cores (D). Mean±SEM (n=3), *, p=0.0171-0.0295; ***, p<0.0001. Magnification, ×4. (E) PC3 cells were treated with vehicle or Gamitrinib as in (A, B) and analyzed for cell proliferation by direct cell counting. Mean±SEM (n=3); ns, not significant. (F) LN229 spheroids were stained with calcein-AM (live cells, bright cluster) and Topro-3 (dead cells, dark spots), and analyzed by 2-photon microscopy. Magnification, ×20. (G) The results of wound closure studies in control and Gamitrinib-treated tumor cells are shown in FIG. 1G. PC3 cells were treated with vehicle or Gamitrinib (5 μM) and analyzed in a wound closure assay after 16 or 24 h. Representative micrographs (top) and quantification of cell motility (bottom) are shown. The leading front of cell migration is indicated by dotted lines. Mean±SEM (n=3), ***, p<0.0001-0.0004. Magnification, ×5. (H) The experimental conditions are the same as in (G) except that normal human MRC-5 lung fibroblasts treated with vehicle or two concentrations of Gamitrinib (5-10 μM) were analyzed in a wound closure assay after 16 or 24 h. Magnification, ×10. AU, arbitrary units.

FIG. 2A through 2F show mitochondrial chaperone TRAP-1 regulation of tumor cell motility. (A, B) The indicated tumor cell lines were transfected with control non-targeting (Ctrl) or TRAP-1-directed pooled siRNA and analyzed by Western blotting (A), or cell migration (B). Top, phase contrast microscopy. Bottom, quantification of cell migration. Mean±SEM (n=3). ***, p<0.0001. (C) The indicated tumor cell types were transfected with control siRNA (Ctrl) or siRNA-directed to TRAP-1 and analyzed for cell proliferation by direct cell counting. Mean±SEM (n=3). (D) the 38 indicated tumor cell lines were transfected with control non-targeting siRNA (Ctrl) or individual siRNA sequences to TRAP-1 and analyzed by Western blotting. (E, F) The indicated tumor cell types were transfected with control siRNA (Ctrl), individual siRNA sequences (#1-4) to TRAP-1 or pooled siRNA (P1) to TRAP-1, and analyzed for cell migration (E) or invasion (F) after 6 or 16 h, respectively. Mean±SEM (n=3). ***, p<0.0001.

FIG. 3A through 3L show cellular requirements of mitochondrial Hsp90-directed tumor cell migration. (A) Representative images from video-sequences of FBS-stimulated PC3 cells treated with vehicle or Gamitrinib (Gam, 5 μM), and analyzed by real-time quantification of membrane ruffling (lamellipodia growth and retraction) by stroboscopic imaging. Bar=16.2 μm length. (B) Stroboscopic images representing an area of analysis (white line in A) for the indicated time intervals. (C) Quantification of membrane ruffling frequency. Each bar corresponds to an individual cell. Broken lines, average values. (D) Average ruffling frequency (top), and quantification of speed of lamellipodia retraction in μm/min (bottom). Mean±SEM (n=15). ***, p<0.0001. (E, F) Tumor cells treated with (+) or without (−) Gamitrinib (Gam, 5 μM) were analyzed by Western blotting. p, phosphorylated. (G) Serum-starved A549 cells were stimulated with EGF (100 ng/ml, 2 min) or FBS (10%, 5 min), treated with (+) or without (−) Gamitrinib (5 μM), and analyzed for GTP-bound Rac1 or Cdc42. (H)PC3 cells were transfected with vector or cDNA encoding FAK and analyzed by Western blotting. p, phosphorylated. (I, J), PC3 cells transfected as in (H) and treated with (+) or without (−) Gamitrinib (5 μM) were analyzed for cell migration (left) or invasion (right) (I), or cell proliferation (J). Mean±SEM (n=3), ***, p<0.0001; ns, not significant. (K, L) PC3 cells were transfected with vector or cDNAs encoding Src (K) or constitutively active Cdc42V12 mutant (L), and analyzed by Western blotting (left) or 39 cell migration (right) in the presence (+) or absence (−) of Gamitrinib. Mean±SEM (n=3). **, p=0.056.

FIG. 4A through 4K show metabolic stress control of tumor cell invasion. (A) PC3 cells were treated with Gamitrinib (Gam, 5 μM), 17-AAG (5 μM), CCCP (12.5 μM) or 2-DG (25 mM) and analyzed for cell invasion. Left, representative image of stained nuclei. Right, quantification. Mean±SEM (n=3). ***, p<0.0001. (B) PC3 cells were treated with Gamitrinib (Gam, 1.25-5 μM) or CCCP (12.5 μM) and analyzed by Western blotting. p, phosphorylated. (C)PC3 cells were treated as in (A) and analyzed for cell proliferation. Mean±SEM (n=3); ns, not significant. (D) LN229 cells were transfected with control siRNA (Ctrl) or TRAP-1-directed siRNA and analyzed by Western blotting with or without glucose (Glc). (E) LN229 cells were treated with vehicle or Gamitrinib (Gam, 5 μM) and analyzed by Western blotting with or without glucose (Glc). (F) LN229 cells were transfected with control (Ctrl) or TRAP-1-directed siRNA, and analyzed for ATP production in the presence of the indicated glucose (Glc) concentrations. LU, luciferase units. Mean±SEM (n=3). (G) LN229 cells were treated with vehicle or Gamitrinib (Gam, 5 μM), and analyzed for cell viability. Bars correspond to conditions of 100% glucose (Glc)-0% galactose (Gal); 50% Glc-50% Gal or 0% Glc-100% Gal. Mean±SEM (n=3), ***, p=0.0007. (H) NIH3T3 fibroblasts were transfected with vector or TRAP-1 cDNA, and analyzed by Western blotting. (I) The experimental conditions are as in (H) except that control or TRAP-1 transfected cells were analyzed for ATP production at the indicated glucose (Glc) concentrations. LU, luciferase units. Mean±SEM (n=3). (J) NIH3T3 fibroblasts transfected as in (H) were analyzed by DAPI staining of invading cells under the indicated conditions. Right, quantification of cell invasion. Mean±SEM (n=3). **, p=0.0029; ***, p<0.0001. (K) NIH3T3 fibroblasts transfected as in (H), were analyzed for cell proliferation. Mean±SEM (n=3); ns, not significant.

FIG. 5A through 5K show LKB1-AMPK regulation of tumor cell motility. (A, B) LN229 cells were transfected with individual siRNA sequences to AMPK (A) or LKB1 (B), and analyzed by Western blotting (top), cell migration (A), or invasion (B) with (+) or without (−) of Gamitrinib (Gam, 5 μM). Mean±SEM (n=3). ***, p<0.0001. (C) LN229 cells were transfected with control siRNA or individual siRNA sequences to AMPK or LKB 1 and analyzed for ATP production with (+) or without (−) of Gamitrinib (5-10 μM). Mean of replicates of a representative experiment out of at least three independent determinations. (D, E) Tumor cell types transfected with control or constitutively active AMPK (AMPK^{CA}) cDNA were analyzed by Western blotting (D) or cell invasion (E). Mean±SD (n=3). *, p=0.0295; ***, p<0.001. (F) The experimental conditions are as in (D) except that transfected LN229 or PC3 cells were analyzed for ATP production. LU, luciferase units. Mean±SEM (n=3). (G, H)PC3 cells were treated with rapamycin (Rapa, 0.1 μM) or Gamitrinib (Gam, 5 μM), and analyzed by Western blotting for differential phosphorylation (p) of the indicated kinases (G), or cell migration after 6 h (H). Mean±SD (n=3). (I, J) The indicated tumor cell lines were transfected with control siRNA (Ctrl) or atg5-directed siRNA and analyzed by Western blotting (I) or cell invasion (J). Mean±SEM (n=3). **, p<0.001. (K) PC3 cells were transfected with control non-targeting siRNA (Ctrl) or TRAP-1-directed siRNA alone or in combination with siRNA sequences directed to AMPK, atg5 or ULK1 and analyzed for cell migration. Mean±SEM (n=3). **, p=0.0022.

FIG. 6A through 6G show ULK1 control of tumor cell motility. (A) PC3 cells were transfected with vector or constitutively active AMPK cDNA (AMPK^{CA}) and analyzed by Western blotting for changes in phosphorylation (p) of the indicated kinases. (B) PC3 cells were treated with vehicle, rapamycin (Rapa, 0.1 μM for 1 h), metformin (5 mM for 6 h) or Gamitrinib (5 μM for the indicated time intervals), and analyzed by Western blotting. p, phosphorylated; veh, vehicle. (C)PC3 cells were treated with 17-AAG (5 μM) for the indicated time intervals and analyzed by Western blotting. (D) PC3 cells transfected with control (Ctrl) or ULK1-directed siRNA were treated with (+) or without (−) Gamitrinib (Gam, 5 μM) and analyzed by Western blotting for differential phosphorylation of the indicated kinases. (E) LN229 cells were transfected as in (D) and analyzed for cell migration (left) or invasion (right) in the presence (+) or absence (−) of Gamitrinib. Mean±SEM (n=3). ***, p<0.0001. (F) PC3 cells transfected with control siRNA (Ctrl) or ULK1-directed siRNA were reconstituted with siRNA-insensitive WT ULK1, kinase-inactive (KI) ULK1, or non-AMPK phosphorylatable ULK1 (4SA) cDNA, and analyzed by Western blotting (top), or cell invasion (bottom). Mean±SEM (n=3). **, p<0.001; ***, p<0.0001. (G) The experimental conditions are as in (F), except that transfected and reconstituted PC3 cells were analyzed for cell proliferation by direct cell counting. Mean±SEM (n=3).

FIG. 7A through 7H show FIP200 regulation of tumor cell motility by bioenergetics stress. (A) PC3 cells were transfected with vector or FIP200 cDNA and analyzed by Western blotting (top) or cell invasion (bottom). Mean±SEM (n=3). ***, p<0.0001. (B) The experimental conditions are as in (A) except that transfected PC3 cells were analyzed for cell proliferation by direct cell counting. Mean±SEM (n=3). (C)PC3 cells were transfected with control siRNA (Ctrl) or the indicated individual siRNA sequences to FIP200 and analyzed by Western blotting (top) or cell invasion (bottom) in the presence (+) or absence (−) of Gamitrinib. Mean±SEM (n=3). ***, p<0.0001. (D) PC3 cells were transfected with control siRNA or FIP200-directed siRNA and analyzed by Western blotting for changes in phosphorylation (p) of the indicated kinases in the presence (+) or absence (−) of Gamitrinib. (E) PC3 cells were treated with vehicle or Gamitrinib (5 μM), and analyzed for gel mobility shift by phosphate-affinity SDS polyacrylamide gel electrophoresis followed by Western blotting using antibodies to FIP200 (left) or pan-phosphorylated Ser residues (right). Phosphorylated isotypes of FIP200 are indicated with an arrow. (F) PC3 cells were transfected with control siRNA (Ctrl), FIP200- or TRAP-1-directed siRNA, alone or in combination, and analyzed by Western blotting. (G, H) The experimental conditions are as in (F) except that transfected PC3 cells were analyzed for cell migration (G) or cell proliferation by direct cell counting (H) in the presence (+) or absence (−) of TRAP-1 silencing by siRNA. Mean±SEM (n=3). ***, p<0.0001.

FIG. 8A through 8I show metabolic control of metastasis. (A) Representative image of single GFP-labeled breast adenocarcinoma MDA-231 cells (arrows) lodged near the growth cartilage of the femora and tibiae of inoculated CB 17 SCID mice. Magnification, ×100 (left). (B) Quantification of bone homing of GFP-labeled MDA-231 cells transfected with control siRNA or TRAP-1-directed siRNA. Mean±SEM (5 animals/group). *, p=0.034. None, untransfected cells. (C) Representative histologic image of livers from animals injected intrasplenically with H460 cells transfected with vector, AMPK^{CA} or ULK1 cDNA. Magnification, ×1. (D) Quantification of number of metastatic foci (top) and metastatic surface area (bottom) per each condition tested. Mean±SEM (3-4 animals/group). ***, p<0.0001; **, p=0.009. (E) Representative images of immunohistochemical expression of ULK1-Ser555 or ULK1-Ser757 in NSCLC TMA sections. Middle panels, representative sections of normal lung weakly positive (<10% stained cells) for ULK1-Ser757 expression. (F, G) Summary of ULK1-Ser555 (F) or ULK1-Ser757 (G) immunoreactivity in NSCLC patients. The analysis parameters are as follows: ULK1-Ser555 (F), n=173; positive, 98 (AdCa), 40 (SCC); negative, 20 (AdCa), (SCC); ULK1-Ser757 (cytosolic reactivity) (G), n=178; positive, 38 (AdCa), 5 (SCC); negative, 83 (AdCa), 52 (SCC). (H, I) Kaplan-Meier survival curves of NSCLC patients according to expression of Ser757-phosphorylated ULK1 (H) or differential ratio (cutoff, 0.2) of Ser757/Ser555-phosphorylated ULK1 (I).

FIGS. 9A and 9 B are schematic representations of bioenergetics stress control of tumor cell motility. (A) Subcellularly-directed inhibition of mitochondrial Hsp90-directed protein folding by Gamitrinib targeting of mitochondrial Hsp90s induces a bioenergetics imbalance due to detachment of HK-II from the mitochondrial outer membrane. In turn, this results in decreased ATP production, activation of the energy sensor LKB-1-AMPK kinase cascade, and AMPK-dependent phosphorylation of ULK1, a component of the upstream autophagy initiator complex. Active ULK1 mediates phosphorylation and activation of FIP200, which in turn inhibits FAK-directed tumor cell motility and metastasis. (B) Despite limited nutrient availability mitochondrial Hsp90s maintain HK-II tethered to the organelle outer membrane and potentially maintain a residual mitochondrial oxidative phosphorylation capacity. In turn, energy produced under these conditions is sufficient to prevent activation of LKB1-AMPK signaling, blocking the formation of a phosphorylation-dependent, active ULK1-FIP200-atg13 complex. This prevents the initiation of autophagy and releases FAK from the inhibitory effect of phosphorylated FIP200, promoting cell motility and metastasis. Gam, Gamitrinib; p, phosphorylation; PTP, permeability transition pore; cyto c, cytochrome c.

FIGS. 10A and 10B show control of cell motility by mitochondrial Hsp90s. (A, B) the indicated tumor cell types were treated with vehicle or Gamitrinib (5 μM) and analyzed for cell migration (A) or invasion (B) after 6 or 16 h, respectively. Mean±SD (n=3). ***, p<0.0001.

FIG. 11 shows mitochondrial Hsp90 regulation of the actin cytoskeleton. The indicated tumor cell types were treated with vehicle or Gamitrinib (5 μM), stained with rhodamine-phalloidin and analyzed by confocal microscopy. Magnification, ×1000.

FIG. 12A through 12D show real-time single cell analysis of cytoskeletal dynamics by mitochondrial Hsp90s. (A) Still images of time lapse video microscopy of individual LN229 cells treated with vehicle or Gamitrinib (5 μM). Individual subcellular areas selected for SACED analysis are depicted as vertical and horizontal lines (4 lines per cell). (B) Stroboscopic images depicting the kinetics of membrane ruffling (lamellipodia growth and retraction) in vehicle- or Gamitrinib-treated LN229 cells. (C) Quantification of membrane ruffling frequency in vehicle- or Gamitrinib-treated LN229 cells. Each bar corresponds to an individual cell. Average values are indicated by dotted lines. (D) The indicated tumor cell types were transfected with vector or constitutively active RacV12 mutant cDNA and analyzed for cell invasion with (+) or without (−) Gamitrinib. Mean±SD (n=3).

FIG. 13A through 13C show mitochondrial Hsp90-directed bioenergetics controls tumor cell migration under stress. (A, B) PC3 cells treated with Gamitrinib (5 μM), 17-AAG (5 μM), 2-DG (25 mM) or CCCP (12.5 μM) were analyzed for cell migration after 6 h by phase contrast microscopy (A), and quantified (B). Mean±SEM (n=3). ***, p<0.0001. (C)PC3 cells were treated as in (A) and analyzed for changes in cell viability by Trypan blue exclusion. Mean±SEM (n=3); ns, not significant.

FIGS. 14A and 14B show mitochondrial Hsp90s-directed bioenergetics under stress conditions. (A) NIH3T3 fibroblasts were transfected with vector or TRAP-1 cDNA, maintained in normal growth conditions (None) or suspended in 5 mM glucose (Glc) or 50% concentrations of amino acids (AA), and analyzed for cell viability by MTT. AU, arbitrary units. (B) The experimental conditions are as in (A), except that control or TRAP-1 transfectants were analyzed for cell viability by Trypan blue exclusion. Mean±SD (n=3).

FIG. 15A through 15C show ULK1-FIP200 regulation of tumor cell motility. (A) The indicated tumor cell types were transfected with vector or constitutively active AMPK 1 cDNA (AMPK^{CA}) and analyzed for cell migration. Mean±SD (n=3). **, p<0.001. (B) PC3 cells were transfected with control siRNA (Ctrl) or siRNA directed to AMPK, ULK1 or atg5, alone or in combination with TRAP-1-directed siRNA, and analyzed by Western blotting. (C)PC3 cells were transfected with control (Ctrl) or ULK1-directed siRNA, and analyzed by Western. (D) PC3 cells transfected with vector or FIP200 cDNA were analyzed for cell migration after 6 h. Mean±SEM (n=3). ***, p<0.0001.

FIGS. 16A and 16B show FIP200 regulation of tumor cell motility. (A, B) PC3 cells were transfected with control (Ctrl) or FIP200-directed siRNA and analyzed by Western blotting (A) or cell migration (B, left) or cell invasion (B, right) in the presence (+) or absence (−) of Gamitrinib. Mean±SD (n=3). ***, p<0.0001.

FIG. 17A through 17F show differential ULK1 phosphorylation influences disease outcome in NSCLC patients. (A) Expression of total ULK1 protein in the NSCLC patients series analyzed in this study. AdCa, adenocarcinoma; SCC, squamous cell carcinoma. (B) Overall survival of NSCLC patients with positive or negative expression of total ULK1 protein. (C) Overall survival of NSCLC patients according to positive (Pos) or negative (Neg) expression of Ser757-phosphorylated ULK1 in cytoplasm (C) or nuclei (N). (D) Preferential expression of Ser757-phosphorylated ULK1 in lung AdCa compared to SCC. N, nuclear; C, cytoplasmic. (E) Correlation between expression of Ser757-phosphorylated ULK1 and NSCLC stage or lymph node metastasis. N0, no lymph node metastasis; N+, one or more lymph node metastasis; T, tumor size, 1-2 or 3-4. N, nuclear; C, cytoplasmic. (F) Correlation between expression of Ser757/Ser555-phosphorylated ULK1 ratio (cutoff, 0.2) and tumor size (T1-2 versus T3-4).

### DETAILED DESCRIPTION OF THE INVENTION

As described herein and exemplified by the data in the examples below, the inventors have determined that interference with Hsp90-directed protein folding in mitochondria triggers cellular starvation, with decreased ATP production and activation of the energy sensor LKB 1-AMPK kinase axis (i). In turn, active AMPK phosphorylates its downstream target and autophagy initiator, ULK1 (ii). Third, activated ULK1 maintains focal adhesion kinase (FAK) under inhibition by the autophagy regulator, FIP200, thus suppressing tumor cell motility (iii). This places changes in the ATP/AMP ratio as the pivotal upstream requirement of this response, and the activation of the ULK1 autophagy regulator as the downstream effector that blunts FAK-dependent cell motility. AMPK activation provides a strong barrier against tumor cell motility and metastasis, in a pathway reversed by mTORC1 activation. FIG. 9 presents a model that summarizes the pathway. The inventors determined that AMPK, ULK1 and atg5 are required for inhibition of cell migration.

In one aspect, therefore, a method for inhibiting tumor cell migration in a mammalian subject comprises reducing or suppressing focal adhesion kinase (FAK) activity, expression or phosphorylation of FAK in a cancer or tumor cell. In another aspect, a method for inhibiting tumor cell migration in a mammalian subject comprises reducing or suppressing ULK1 kinase activity or expression or reducing or suppressing phosphorylation of ULK1 on Ser757 in a cancer or tumor cell. In another aspect, a method for inhibiting tumor cell migration in a mammalian subject comprises reducing or suppressing activation, expression or signaling of the mTORC1 (Ser757) pathway in a cancer or tumor cell. In still another aspect, a method for inhibiting tumor cell migration in a mammalian subject comprises activating or increasing activity or expression of AMPK or LKB 1 or FIP200, or increasing the phosphorylation of FIP200. In still another aspect, a method for inhibiting tumor cell migration in a mammalian subject comprises reducing, suppressing or silencing the activation or expression of TRAP1.

In another aspect, a method for inhibiting tumor cell migration in a mammalian subject comprises administering to a subject a therapeutically effective dose of a composition comprising a molecule that reduces or suppresses focal adhesion kinase (FAK) activity or expression or phosphorylation of FAK in a cancer cell. In one embodiment, the composition may block or mutate the Tyr 397 site of FAK. In another embodiment the composition may block or mutate the Tyr925 site of FAK. Blockage of either site could prevent or reduce phosphorylation at that site. In another aspect, a method for inhibiting tumor cell migration in a mammalian subject comprises administering to a subject a therapeutically effective dose of a composition comprising a molecule that reduces or suppresses ULK1 kinase activity or expression. In another aspect, a method for inhibiting tumor cell migration in a mammalian subject comprises administering to a subject a therapeutically effective dose of a composition comprising a molecule that suppresses activation, expression or signaling of the mTORC1 (Ser757) pathway. In yet another aspect, a method for inhibiting tumor cell migration in a mammalian subject comprises administering to a subject a therapeutically effective dose of a composition comprising a molecule that activates or stimulates AMPK or LKB1 or its activity or expression. In yet another aspect, a method for inhibiting tumor cell migration in a mammalian subject comprises administering to a subject a therapeutically effective dose of a composition comprising a molecule that reduces, suppresses or silences TRAP1, or its activity or expression.

In one embodiment of the methods described above, the composition administered to the subject suppresses phosphorylation of ULK1 on Ser757. In a certain embodiment, as demonstrated in the examples below, this method is useful in subjects with lung cancer. Similarly in another aspect, the composition that suppresses mTORC1 activation is similarly useful in the treatment of subjects with lung cancer. It is further found that compositions that can be employed to adjust the ratio of the AMPK pathway to mTORC1 pathway in favor of the AMPK pathway are further useful in retarding tumor mobility.

As used herein, the term “subject” as used herein means a multicellular and/or mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, and others.

The term “neoplastic disease”, “cancer” or “proliferative disease” as used herein refers to any disease, condition, trait, genotype or phenotype characterized by unregulated or abnormal cell growth, proliferation or replication. The abnormal proliferation of cells may result in a localized lump or tumor, be present in the lymphatic system, or may be systemic. In one embodiment, the neoplastic disease is benign. In another embodiment, the neoplastic disease is pre-malignant, i.e., potentially malignant neoplastic disease. In a further embodiment, the neoplastic disease is malignant, i.e., cancer. In still a further embodiment the neoplastic disease may be caused by viral infection.

In one embodiment, the neoplastic disease is an epithelial cancer. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, leukemia, myeloma, lymphoma, glioma, and multidrug resistant cancer. In another embodiment, the neoplastic disease is Kaposi's sarcoma, Merkel cell carcinoma, hepatocellular carcinoma (liver cancer), cervical cancer, anal cancer, penile cancer, vulvar cancer, vaginal cancer, neck cancer, head cancer, multicentric Castleman's disease, primary effusion lymphoma, tropical spastic paraparesis, adult T-cell leukemia, Burkitt's lymphoma, Hodgkin's lymphoma, post-transplantation lymphoproliferative disease, nasopharyngeal carcinoma, pleural mesothelioma (cancer of the lining of the lung), osteosarcoma (a bone cancer), ependymoma and choroid plexus tumors of the brain, and non-Hodgkin's lymphoma. In still other embodiments, the cancer may be a systemic cancer, such as leukemia. In one aspect, as exemplified, the cancer is a human glioblastoma. In another aspect, the cancer is a prostate adenocarcinoma. In still another embodiment, the cancer is a lung adenocarcinoma. In one embodiment, the cancer is non-small cell lung adenocarcinoma (NSCLC). In another embodiment, the cancer is squamous cell carcinoma. In another embodiment, the cancer is liver cancer. In another embodiment, the cancer is a breast adenocarcinoma. In still another exemplified embodiment, the cancer is melanoma.

The term “benign” condition as used herein refers to a condition which is not a neoplastic disease, i.e., the benign condition is not cancer. In one embodiment, the benign condition is a wart, such as common warts, plantar warts, subungual warts, or periungual warts, among others. In yet a further embodiment, the benign condition is respiratory papillomatosis or epidermodysplasia verruciformis. Still other benign conditions caused by uncontrolled cell proliferation are included herein.

In the performance of the methods described above and exemplified by the data in the examples and figures, the method of inhibiting tumor mobility or metastasis can be practiced when the subject has an established malignancy or refractory cancer. Similarly such methods are useful when the subject is newly diagnosed and prior to treatment. These methods in another embodiment involve administration of one or more effective compositions at a selected time during the course of cancer therapy. In still another embodiment, the methods are used as part of a protocol of neoadjuvant therapy.

In one exemplary embodiment, a composition having one or more of the effects described herein is Gamitrinib. As used herein, the term “Gamitrinib” refers to any one of a class of geldanamycin (GA)-derived mitochondrial matrix inhibitors. Gamintrinibs contain a benzoquinone ansamycin backbone derived from the Hsp90 inhibitor 17-(allylamino)-17-demethoxygeldanamycin (17-AAG), a linker region on the C17 position, and a mitochondrial targeting moiety, either provided by 1 to 4 tandem repeats of cyclic guanidinium (for example, a tetraguanidinium (G4), triguanidinium (G3), diguanidinium (G2), monoguanidinium (G1),) or triphenylphosphonium moiety (Gamitrinib-TPP-OH). For example, Gamitrinib-G4 refers to a Gamitrinib in which a tetraguanidinium moiety is present. For example, Gamitrinib-TPP refers to a Gamitrinib in which a triphenylphosphonium moiety is present. Also throughout this application, the use of the plural form “Gamitrinibs” indicates one or more of the following: Gamitrinib-G4, Gamitrinib-G3, Gamitrinib-G2, Gamitrinib-G1, and Gamitrinib-TPP or Gamitrinib-TPP-OH. Gamitrinib is a small molecule inhibitor of Hsp90 and TRAP-1 ATPase activity, engineered to selectively accumulate in mitochondria. In a preferred embodiment, the Gamintrinib is Gamitrinib-TPP-OH. See, e.g., United States Patent Publication No. 2009/0099080, which is hereby incorporated by reference in its entirety.

However, these methods are not limited to Gamitrinib and it is anticipated that additional compositions, even small molecule compositions having the desired biological effects identified herein may similarly be employed in these methods. For example, among such compositions are known and/or newly developed ligands, such as antibodies, antibody fragments, and synthetic molecules incorporating antibody fragments and CDRs, directed to the targets, TRAP1, FAK, ULK1, mTORC1, LKB1, FIP200 or AMPK. As used herein, the term “antibody,” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), diabodies, Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

Also useful in suppressing or silencing are “antisense” nucleotide sequence or a small nucleic acid molecule having a complementarity to a target nucleic acid sequence, e.g., TRAP1, FAK, ULK1, mTORC1, AMPK, FIP200 or LKB1. It can also comprise a nucleic acid sequence having complementarity to a sense region of the small nucleic acid molecule. For example, in one embodiment the composition comprises a nucleic acid construct comprising a sequence that reduces or suppresses the expression of FAK or ULK1, TRAP1 or mTORC1 or a combination thereof in the target cancer cells. For example, the down regulating composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence.

Where the goal is enhancing expression of the target molecule, e.g., AMPK or LKB1 or FIP200, the composition can be a nucleic acid construct comprising a sequence encoding AMPK or LKB1 or FIP200 under the regulatory control of a promoter that overexpresses or can overexpress the AMPK or LKB1 or FIP200 sequence in the target cancer or tumor cells. For example, the nucleic acid construct can include a viral vector or plasmid vector containing which has one or more iterations of the AMPK or LKB1 or FIP200 sequence under the control of a strong constitutive or inducible promoter so that the expression of the AMPK, LKB 1 or FIP200 RNA is overexpressed in the target cancer cells.

Similarly the methods of this invention may employ such compositions directed to other molecules in the pathways described herein.

In still other embodiments, combinations of Gamitrinib with one or more of these other compositions, e.g., antibodies to one or more of the targets described above, may be prepared for simultaneous or sequential administration to a subject in need thereof. Still other combinations may include Gamitrinib or an antibody to one of the other targets (e.g., FAK, ADPK, LKBI, FIP200, mTORC1, TRAP1 ULK1) with a mitrochondrial uncoupler, such as carbonyl cyanide 3-chlorophenylhydrazone (CCCP). Still other combinations may include Gamitrinib or an antibody to one of the other targets (e.g., FAK, ADPK, mTORC1, TRAP1 ULK1) with a non-hydrolysable glucose analog, such as 2-deoxyglucose.

Desirably, the methods further involve, in one aspect, administering a low dose or suboptimal dose of the composition prior to or during a course of chemotherapy or radiation to reduce the size of an existing primary tumor. In another embodiment, the methods involve administering a suboptimal dose of the composition prior to or during surgery performed to debulk or remove a primary tumor. In still another embodiment, the methods comprise administering the suboptimal dose of the composition after surgery performed to debulk or remove a primary tumor. In yet a further embodiment, the methods involve administering the suboptimal dose of the composition prior to or during a second or repeated course of chemotherapy or radiation. In certain embodiments, the second or repeated course is post-surgery. Still further embodiments of the methods described herein include administering a continuous course of a suboptimal a dose of the composition to a subject in need thereof from prior to a first round of pre-surgical chemotherapy, and/or during and after surgery, and/or prior to and after a second round of post-surgical chemotherapy. The neoadjuvant protocol can further or alternatively involve administering periodic suboptimal doses of the composition to a subject in need thereof. The suboptimal doses are administered at suitable intervals prior to a first round of pre-surgical chemotherapy or radiation, after surgery, and prior to and after a second or repeated course of post-surgical chemotherapy or radiation.

By use of the term “suboptimal dose” is meant the lowest dose of the composition, e.g., Gamitrinib or other compositions that is effective to suppress FAK kinase activity or inhibit phosphorylation of FAK in tumor cells or suppress ULK1 kinase activity, or suppress activation or signaling of the mTORC1 (Ser757) pathway or activate AMPK, or some combination of any of these effects. Preferably the suboptimal dose does not have a cytotoxic effect on the tumor cells or on healthy cells, but retards tumor cell migration. In other embodiments, the suboptimal dose does not cause systemic immune suppression. In still other embodiments, the suboptimal dose is delivered in a continuous infusion or a slow release formulation. As used herein, a “non-cytotoxic amount” refers to a concentration which is, by itself, insufficient to kill the targeted cell, i.e., the cancer cell or healthy cells. Thus, in one embodiment, a non-cytotoxic amount is a concentration sufficient to produce the above-described effects in tumor cells. The dosage required for a non-cytotoxic amount will depend primarily on factors such as the condition being treated, the age, weight and health of the patient, and may thus vary among patients.

For example, where the composition is or comprises Gamitrinib, one such suboptimal dose is 5 μM Gamitrinib. In another embodiment, the suboptimal dose is less than 5 μM Gamitrinib. In still another embodiment, the suboptimal dose is between 1 μM and 5 μM Gamitrinib. In another embodiment, the suboptimal dose is less than 1 μM Gamitrinib. In still another embodiment, the suboptimal dose is less than 0.5 μM Gamitrinib. Put another way, the suboptimal doses can be less than 3 mg/kg of patient weight, less than 2 mg/kg of patient weight, or less than 1 mg/kg of patient weight.

The therapeutic compositions administered by these methods, e.g., either the exemplary Gamitrinib or other compositions, are administered directly into the environment of the targeted cell undergoing unwanted proliferation, e.g., a cancer cell or targeted cell (tumor) microenvironment of the patient. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically.

These therapeutic compositions may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

The compositions are administered in sufficient amounts inhibit migration of the cancer cells through the basement membrane and provide a therapeutic benefit without undue adverse or with medically acceptable physiological effects, which can be determined by those skilled in the medical arts. Methods for determining the timing of frequency of administration will include an assessment of tumor response to the reagent administration.

In still other embodiments, these methods of administering the compositions accomplishing the biological effects are part of a neoadjuvant or combination therapy. In one embodiment, the composition as described above, can be administered alone or in combination with various other treatments or therapies for the proliferative disease, e.g., cancer. In one embodiment, the method further comprises administering to the subject along with the therapeutic agent another adjunctive therapy which may include a monoclonal antibody, chemotherapy, radiation therapy, a cytokine, or a combination thereof.

In still another embodiment the methods herein may include co-administration or a course of therapy also using other small nucleic acid molecules or small chemical molecules or with treatments or therapeutic agents for the management and treatment of the proliferative disease, e.g., cancer. In one embodiment, a method of treatment of the invention comprises the use of one or more drug therapies under conditions suitable for the treatment of that particular cancer type.

In another embodiment of combination therapy, the therapeutic agent is administered at a suboptimal dose that can immediately start eliminating the targeted cell undergoing unrestricted or abnormal replication or proliferation, e.g., tumor. This is accompanied by administration of active immunotherapy to induce an active endogenous response to continue the tumor eradication. In one embodiment, the methods described herein include administration of other known anti-proliferative disease therapies. For example, surgical debulking, in certain embodiments is a necessary procedure for the removal of large benign or malignant masses, and can be employed before, during or after application of the methods and compositions as described herein. Chemotherapy and radiation therapy, in other embodiments, bolster the effects of the methods described herein. Finally, immune-based therapies can eradicate residual disease and activate endogenous antitumor responses that persist in the memory compartment to prevent metastatic lesions and to control recurrences. Such combination approaches (surgery plus chemotherapy/radiation plus immunotherapy) are anticipated to be successful in the treatment of many proliferative diseases along with the methods described herein.

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### Example 1

### Materials and Methods

Cell Culture.

Human glioblastoma LN229, prostate adenocarcinoma PC3 and PC3-ML subline, lung adenocarcinoma H1299, H1437, H460 and A549, breast adenocarcinoma MDA-MB-231, melanoma 1205Lu and WM793, or normal NIH3T3 and MRC-5 fibroblasts were obtained from the American Tissue Culture Collection (ATCC), and maintained in culture according to the supplier's specifications. For metabolic stress experiments, cells were incubated in MEM-based media containing glucose, essential and non-essential amino acids and vitamins in identical concentration as DMEM, plus 4 mM L-glutamine, 1 mM sodium pyruvate, 10 mM HEPES, and 10% 10 K dialyzed FBS (Gibco). Three conditions were tested: 25 mM glucose (complete medium), 5 mM glucose or 50% amino acid deprivation compared to DMEM. Galactose challenge experiments were performed by culturing the cells in DMEM No Glucose medium supplemented with 4 mM L-glutamine, 10% 10K dialyzed FBS and the indicated mixtures of D-(+)-glucose and D-(+)-galactose to a final concentration of 25 mM.

Protein Analysis.

Protein lysates were prepared in RIPA buffer containing 150 mM NaCl, 1.0% Triton X-100, 0.5% sodium deoxycholate, 0.1% SDS, 50 mM Tris, pH 8.0, in the presence of EDTA-free protease inhibitor cocktail (Roche) and phosphatase inhibitor cocktail 2 and 3 (Sigma Aldrich). Equal amounts of protein lysates were separated by SDS gel electrophoresis, transferred to PVDF membranes and incubated with primary antibodies of various specificities. Protein bands were detected by chemiluminescence, as described (21).

ATP Measurement.

Intracellular ATP concentrations were measured by the luciferin-luciferase method using an ATP measuring kit (Biochain). The ATP concentration in each extract was determined in a microplate luminometer (Beckman Coulter) against standard ATP solutions used as reference.

Animal Models of Skeletal or Liver Metastasis.

All experiments in vivo were carried out in accordance to the Guide for the Care and Use of Laboratory Animals of the National Institutes of Health (NIH). Protocols were approved by an Institutional Animal Care and Use Committee (IACUC). For an animal model of skeletal metastasis (51, 52), five weeks-old female CB17-SCID mice were obtained from Taconic and housed in a germ-free barrier. At 6-8 weeks of age, mice were anesthetized with the combined administration of ketamine (80 mg/kg) and xylazine (10 mg/kg) administered by intraperitoneal route, and then inoculated in the left cardiac ventricle with breast adenocarcinoma MDA-231 cells. Cell inoculation was performed using an insulin syringe with a 30-gauge needle. The correct penetration of the cardiac wall was established by the appearance of fresh arterial blood in the Luer-Lok fitting of the hypodermic needle. In addition, blue-fluorescent polystyrene beads (10 μm diameter, Invitrogen-Molecular Probes) were co-injected with cancer cells and their detection in the adrenal glands was utilized to confirm successful inoculation in the blood circulation (51, 52). Animals were sacrificed after 72 h, and adrenal glands and bones were fixed in 4% formalin for 48 h. Femora and tibiae were decalcified in 0.5 M EDTA for 4 days and tissues were frozen in O.C.T. embedding medium (Electron Microscopy Sciences, Hatfield, Pa.) after a cryoprotection step in 25% sucrose for 24 h. Serial tissue sections of 80 μm in thickness were prepared using a Microm HM550 cryostat (Mikron). Sections of each hind leg and soft-tissue organs were transferred on glass slides, stored at −20° C. and examined for cancer cells using a Zeiss AX10 fluorescence microscope connected to a Nuance multispectral imaging system (CRI) with a measurement module included in the analysis software (v. 2.4). Bright field and fluorescence images were acquired with an Olympus DT70 CCD color camera (51, 52).

For an animal model of liver metastasis (53), six- to eight-weeks old female SCID/beige mice were anesthetized with ketamine hydrochloride, the abdominal cavity was exposed by laparotomy, and injected into the spleen with 4×10⁶ H460 cells previously transfected with control plasmid, ULK1 cDNA or constitutively active AMPK^{CA} cDNA. Spleens were removed the following day to minimize effects on metastasis due to variable growth of primary tumors. Animals were sacrificed at 11 d after injection, and their livers were resected, fixed in formalin and paraffin embedded. Liver sections were stained with hematoxilin and eosin and analyzed histologically. Metastatic foci were quantified in serial tissue sections by histology and expressed as number of lesions and surface areas of tumor growth (53).

Patient Samples.

A series of 180 consecutive patients surgically treated for non-small cell lung cancer (NSCLC) at Fondazione IRCCS Ca' Granda Hospital (Milan, Italy) between 2000 and 2004 was available for this study. This patient series included 123 cases of adenocarcinoma (AdCa) and 57 cases of squamous cell carcinoma (SCC) of the lung. Clinical outcome data were available for all patients. NSCLC cases were staged according to the current TNM classification of malignant tumors (International Union Against Cancer, UICC, 7th edition, 2009). An informed consent was obtained from all patients enrolled, and the study was approved by an Institutional Review Board of the Fondazione IRCCS Ca' Granda, Milan, Italy. The follow-up period ranged from 0 to 132 months (average 55.2 months). At the last follow-up (January 2011), 103 patients were deceased for progression of NSCLC, whereas 77 patients were alive. Patients' characteristics are summarized in Table 1.

TABLE 1

| | CLINICOPATHOLOGICAL | HISTOTYPE | |
|---|---|---|---|
| FEATURE | FEATURE | AdCa (n = 123) | SCC (n = 57) |
| Gender | Male | 91 | 50 |
| | Female | 33 | 7 |
| Age | Median (range) | 62.5 (42-78) | 65.6 (45-82) |
| Grade (G)¹ | G1 | 5 | 1 |
| | G2 | 52 | 23 |
| | G3 | 51 | 26 |
| Tumor size (T) | T1a | 18 | 7 |
| | T1b | 14 | 7 |
| | T2a | 50 | 23 |
| | T2b | 10 | 8 |
| | T3 | 28 | 10 |
| | T4 | 3 | 2 |
| Lymph node metastases | Nx | 6 | — |
| (N) | N0 | 65 | 35- |
| | N1 | 27 | 19 |
| | N2 | 25 | 3 |
| | N3 | — | — |
| Distant metastases (M) | M1a | 5 | 2 |
| Status | Alive | 55 | 22 |
| | Dead | 68 | 35 |

Statistical Analysis.

Data were analyzed using the two-sided unpaired t tests using a GraphPad software package (Prism 4.0) for Windows. For analysis of patient samples, groups were compared using the Student's t tests as univariate statistics. For overall survival analysis, the Kaplan-Meyer method was used. Patients negative for ULK1-Ser757 were plotted separately from ULK1-Ser757-positive cases and the two-sided log-rank test was used to compare the two curves. The phosphorylation event at Ser757 or Ser555 has opposite effects on ULK1 function. When the immunoreactivity of both phosphorylated forms was considered, a score was computed for each sample summing Ser757 immunoreactivity in the cytoplasm and in nuclei and dividing for a Ser555 immunoreactivity value. Kaplan-Meyer curves for patient overall survival (OS) under the various conditions examined were then generated. Data are expressed as mean±SD or mean±SEM of at least three independent experiments. A p value of <0.05 was considered as statistically significant.

Study Approval.

Animal studies were approved by an Institutional Animal Care and Use Committee (IACUC) from The Wistar Institute or Drexel University College of Medicine. For studies using human samples, an informed consent was obtained from all patients enrolled, and the study was approved by an Institutional Review Board of the Fondazione IRCCS Ca' Granda, Milan, Italy.

Antibodies and Reagents.

The following antibodies to Ser473-phosphorylated Akt (Cell Signaling), Akt (Cell Signaling), Thr202/Tyr204-phosphorylated ERK1/2 (Cell Signaling), ERK1/2 (Cell Signaling), Tyr397-phosphorylated FAK (Invitrogen), Tyr925-phosphorylated FAK (Cell Signaling), FAK (Cell Signaling), Tyr416-phosphorylated Src (Cell Signaling), Src (Cell Signaling), Rac1 (Upstate), Cdc42 (Cell Signaling), Ser199/204-phosphorylated Pak1/Ser192/197-phosphorylated Pak2 (Cell Signaling), Ser144-phosphorylated Pak1/Ser141-phosphorylated Pak2 (Cell Signaling), Ser20-phosphorylated Pak2 (Cell Signaling), Pak1/2/3 (Cell Signaling), FIP200 (Novus Biologicals), TRAP-1 (BD Biosciences), HA (Roche), LKB1 (Cell Signaling), Thr172-phosphorylated AMPK (Cell Signaling), AMPK (Cell Signaling), atg5 (Cell Signaling), Ser792-phosphorylated Raptor (Cell Signaling), Raptor (Cell Signaling), Ser2448-phosphorylated mTOR (Cell Signaling), mTOR (Cell Signaling), Thr37/46-phosphorylated 4EBP1 (Cell Signaling), 4EBP1 (Cell Signaling), Ser79-phosphorylated Acetyl-CoA Carboxylase (ACC) (Cell Signaling), ACC (Cell Signaling), Ser555-phosphorylated ULK1 (Cell Signaling), Ser757-phosphorylated ULK1 (Cell Signaling), ULK1 (Santa Cruz), LC-3 (Cell Signaling), pan-phosphorylated-Ser residues (Millipore), β-tubulin (Sigma-Aldrich) and β-actin (Sigma-Aldrich) were used.

The plasmids encoding Cdc42V12 (Addgene #11399), Rac1V12 (Addgene #11397), HA-tagged FIP200 (Addgene #24303), myc-tagged wild type ULK1 (Addgene #27629), myc-tagged kinase inactive (KI) ULK1 (Addgene #27630), myc-tagged ULK1 non-phosphorylatable mutant 4SA (Addgene #27631), constitutively activated AMPK 1 (AMPK^{CA}, 1-312) (Addgene #27632), Src (Addgene #13663) were used. A cDNA encoding FAK or TRAP-1 was cloned into pcDNA6/myc-His (Invitrogen), and the construct was validated by DNA sequencing. The complete chemical synthesis, HPLC profile, and mass spectrometry of mitochondrial-targeted small molecule Hsp90 antagonist, Gamitrinib (GA mitochondrial matrix inhibitors) has been reported previously (24). The Gamitrinib variant containing triphenylphosphonium as a mitochondrial-targeting moiety was used in this study (24). Non-mitochondrially permeable Hsp90 inhibitor 17-allylamino demethoxygeldanamycin (17-AAG) was obtained from LC-Laboratories. 2-deoxy-D-glucose (2-DG) and carbonyl cyanide 3-chlorophenylhydrazone (CCCP) were obtained from Sigma-Aldrich. Rapamycin and metformin were obtained from EMD. Calcein-AM and Topro were from Invitrogen.

Transfections.

Gene knockdown experiments were carried out using control, non-targeting small interfering RNA (siRNA) pool (Dharmacon, cat. no. D-001810) or specific siRNA pools targeting TRAP-1 (Dharmacon, cat. no. L-010104), atg5 (Dharmacon, cat. no. L-004374), LKB1 (Dharmacon, cat. no. L-005035), AMPK α1/α2 (Santa Cruz Biotechnology, cat. no. sc-45312), FIP200 (Dharmacon, cat. no. L-021117), or ULK1 (Santa Cruz Biotechnology, cat. no. sc-44182). Individual ON-Target SMART siRNA were used for TRAP1 (Dharmacon, cat. no. J-010104-05, -06, -07 and -08), LKB1 (Dharmacon, cat. no. J-005035-07, -08, -09 and -10), AMPK α1/α2 (Santa Cruz Biotechnology, cat. no. sc-45312A, B and C), and FIP200 (Dharmacon, cat. no. J-021117-05, -06, -07 and -08).

For gene silencing, pooled or individual siRNA oligonucleotide sequences were transfected at 10-30 nM concentrations in the presence of Lipofectamine RNAiMAX in a 1:1 ratio (Invitrogen). Cells were incubated for 48 h, validated for target protein knockdown by Western blotting, and processed for subsequent experiments. Plasmid DNA transfections were carried out using X-tremeGENE HP DNA transfection reagent (Roche) for PC3 or LN229 cells, or Lipofectamine LTX (Invitrogen) for MDA-231, NIH3T3 or H460 cells. In some experiments, LN229 or PC3 cells were transfected with siRNA directed to human ULK1, incubated for 48 h, and subsequently transfected with siRNA-resistant mouse ULK1 cDNA constructs.

Cell Migration and Invasion.

Various tumor cell types were treated as indicated in each experiment, suspended in 0.1% BSA/DMEM and seeded (1.6−3.2×103 cells/mm2, depending on the cell type) in the upper compartment of 8 μM pore diameter BD transwells (BD). NIH3T3 conditioned medium was placed in the lower compartment as a chemoattractant. After 6-18 h incubations at 37° C., the transwell membranes were recovered and cells on the upper side (non-migratory) were wiped off the surface. Cells on the lower side of the membrane were fixed in methanol, rinsed in water and mounted on glass slides with Vectashield medium containing DAPI (Vector Laboratories). Migrated cells on each membrane were counted by fluorescence microscopy in 5 different fields. For cell invasion assays, transwell membranes were coated with Matrigel and processed as described above.

For cell migration experiments using a wound closure assay, confluent monolayers of MRC-5 cells were incubated with vehicle or Gamitrinib (5-10 μM), and wounded using a 10 μl pipette tip. Three micrographs/well were obtained at time=0, 16 and 24 h after wounding, and the percentage of wound closure was normalized to the maximum initial area for each well. For analysis of tumor cell invasion in 3D organotypic spheroids, tumor cells (5×10⁴) were seeded onto 96-well plates coated with 1.5% agar (Difco Noble Agar) in PBS, pH 7.4. Spheroids were allowed to form over a 72 h period and then embedded in 600 μA of bovine collagen type I (Organogenesis) in 24-well plates. Spheroids were overlaid with 1 ml of growth medium, treated with various concentrations of Gamitrinib for 72 h, and analyzed for changes in maximum invasion distance and invasion area. Quantification of live vs. dead cells under the various conditions tested was performed by staining the spheroids with calcein-AM (live, bright cluster) and Topro-3 (dead, dark spots) (Invitrogen) for 2 h. Samples were imaged using a Prairie Ultima II 2-photon microscope (Prairie Technologies, Inc, Middleton, Wis.), and stacks of 100 slices were generated in 2 channels. 3-D reconstruction of the labeled spheroids and analysis of cell staining was carried out using ImagePro Plus 3D software (Media Cybernetics, Silver Spring Md.).

For analysis of nutrient deprivation, tumor cell types were preincubated in the presence of 50% amino acids or 5 mM glucose for 16 h before seeding for cell migration or cell invasion studies. BSA (0.1%) or dialyzed FBS (10%) were added to the upper and lower compartments of the Transwell chamber, respectively, to maintain the nutrient-deprived conditions throughout the cell motility studies.

Rac1 and Cdc42-GTP Pull-Down Assays.

The activation of Rho family small GTPases, Rac1 or Cdc42 was examined in pull-down assays using the p21-binding domain (PBD, amino acids 70-117) of the p21-activated kinase-1 (Pak1). Briefly, a pGEX TK-Pak1 PBD cDNA (Addgene, Cat. no. #12217) was purified, transformed into BL21 E. coli competent cells (Stratagene), and expressed as recombinant GST fusion protein after induction with 1 mM IPTG for 4 h at 34° C. Cells were suspended in PBS, pH 7.2, in the presence of protease inhibitors (SIGMA), and broken by sonication in 1% Triton X-100 for 30 min at 4° C. Soluble proteins were isolated by chromatography on glutathione Sepharose 4B (GS4B, GE Healthcare), eluted in 3 consecutive steps in buffer containing 10 mM GSH, 50 mM Tris HCl, pH 8.0, and further desalted using Amicon Ultra 4/10K columns (Millipore), for a total of 3 buffer changes to PBS, pH 7.2. The protein was diluted in glycerol, quantified by absorbance at 280 nm, and stored at −80° C. until use. The activity of each batch of recombinant protein was assessed by incubating aliquots of the cell lysate with non-hydrolyzable GTPγS at 0.1 mM (maximum binding) or 1 mM GDP (negative control) in low Mg2+ buffer at 30° C. followed by pull-down (54).

For modulation of small GTPase activity, tumor cells at 30% confluency were serum-starved for 48 h, stimulated with FBS (10% for 5 min) or EGF (100 ng/ml for 2 min), and lysed in pull-down buffer containing 20 mM Tris-HCl, pH 7.4, 150 mM NaCl, 5 mM MgCl2, 0.5% NP-40, 5 mM β-glycerophosphate, 1 mM DTT plus protease inhibitors in the presence of 15 μg/ml of GST-Pak1 PBD. Lysates were cleared by centrifugation at 13,000 g for 10 min at 4° C., and incubated with GS4B beads for additional 45 min at 4° C. After centrifugation, GS4B-bound proteins were washed twice in pull-down buffer, separated by electrophoresis on SDS polyacrylamide gels, and Rac1 and Cdc42 levels in pellets or whole cell lysates were analyzed by Western blotting.

F-Actin Staining.

Tumor cells growing at low confluency (1-2×10⁴/well) on optical grade glass coverslips were treated with vehicle or Gamitrinib (5 μM), fixed in 4% formaldehyde for 15 min at 37° C., washed in PBS, pH 7.4, and permeabilized with 0.1% Triton X-100 for 5 min at 22° C. Slides were washed in PBS, pH 7.4, blocked in 1% BSA/PBS for 30 min, incubated with 1:2000 dilution of phalloidin-rhodamine (Molecular Probes) for 30 min, washed in PBS, pH 7.4, and mounted in Fluoromount G medium (Southern Biotech). Slides were analyzed on a Leica TCS SP2 confocal laser microscope with a 100× oil objective.

Quantification of Lamella Dynamics in Live Cells.

Tumor cells growing at low confluency (3-5×10⁴/well) on high optical quality 96 well μ-plates (Ibidi) were imaged with a 40× objective on a Nikon TE300 inverted time-lapse microscope equipped with a video system containing a Evolution QEi camera and a time-lapse video cassette recorder. All experiments were carried out in atmosphere-equilibrated environment at 37° C. and 5% CO₂. Phase contrast images were captured at 0.5 sec intervals for 5 min (600 images=300 sec) and merged into sequence files using ImagePro Plus 7. Real time dynamics of a particular cellular region were quantified by Stroboscopic Analysis of Cell Dynamics (SACED) (55), with generation of digital steps from the first 120 sec (240 frames) of the sequence files imported into Image J software. A particular region of 16.2 μm×0.162 μm (“SACED line”) was selected in cells under analysis, duplicated and montaged in sequence to display the region over time in a stroboscopic image. This process was repeated to obtain a total of 4 SACED lines and therefore 4 stroboscopic images per each cell, which were separately displayed to quantify lamella dynamics. Structures such as protruding lamellipodia and ruffles were manually labeled and the frequency of ruffles per min was calculated. Mean values were calculated from at least 15 cells from 4 separate wells. All experiments were repeated at least twice with different tumor cell types.

Detection of FIP200 Phosphoisotypes.

Analysis of phosphoisotypes was carried out by standard phosphate-affinity SDS gel electrophoresis, using a mobility shift protocol (56). Briefly, proteins were separated by electrophoresis on 3% polyacrylamide/0.5% SeaKem Gold Agarose (Lonza) gels containing the dinuclear metal complex Mn2+-Phos-tag (Acrylamide-pendant Phos-tag, Wako Chemicals). After removal of Mn2+ by washing the gels in blotting buffer containing 1 mM EDTA, proteins were transferred to PVDF membranes and detected by Western blotting using antibodies to FIP200 or pan-phosphorylated Ser residues.

Cell Viability and Cell Proliferation Assays.

Tumor cells were plated onto 96-well plates at 3.7×10³/well and treated with vehicle or Gamitrinib (5 μM). After 24 h, cell viability was assessed with a 3 (4,5-dimethyl-thyazoyl-2-yl)2,5 diphenyltetrazolium bromide (MTT) colorimetric assay (24), or, alternatively, by Trypan blue exclusion and light microscopy. Data were background-subtracted relative to vehicle-treated cultures. In some experiments, tumor cell proliferation was assessed by direct cell counting and light microscopy.

Tissue Microarray (TMA).

Representative tissue blocks from patients affected by NSCLC were used to build seven TMAs (NSCLC-TMA), as described previously (57). For each patient, four cores of neoplasia were included in the blocks as well as 34 cores of non-neoplastic lung parenchyma. For quality control, a 4-μm-thick section was cut from each TMA block, stained with H&E, and analyzed by immunohistochemistry.

Immunohistochemistry.

NSCLC-TMA slides were subject to antigen retrieval in EDTA solution. Sections (4-μm thick) were cut from all TMA blocks and stained with a rabbit monoclonal antibody to phospho-ULK1 Ser555 (1:500, clone D1H4), or to phospho-ULK1 Ser757 (1:100) overnight at 4° C. or for 30 min at 22° C., respectively. Immunohistochemistry (IHC) was performed using a Ventana BenchMark Ultra autostainer (Ventana Medical Systems), with the ultraView Universal DAB Detection Kit (Ventana) for detection of antibody reactivity. All slides were counterstained with hematoxylin. Immunoreactivity for the various markers was evaluated by two pathologists and independently scored for cytoplasmic or nuclear localization. The percentage of immunoreactive epithelial cells was recorded and when discrepancies in scoring occurred, a consensus interpretation was reached after re-examination. Among the lung AdCa samples in the series under investigation, 20 cases were negative for Ser555 expression, whereas 15 cases of SCC were Ser555-negative. The ULK1-Ser757 immunoreactivity was detected in both cytoplasm and nuclei. The number of immunoreactive or negative cases per phospho-ULK1 protein is summarized in Table 2. Ninety-two AdCa and 37 SCC patients could be analyzed for both 5555 and 5757 immunoreactivity.

TABLE 2

| | | NSCLC | | |
|---|---|---|---|---|
| Antibody | Localization | histoype | Positive IHC | Negative IHC |
| Ser555² | Nuclear | AdCA | 98 | 20 |
| Ser757³ | | SCC | 40 | 15 |
| | Nuclear | AdCa | 74 | 49 |
| | | SCC | 2 | 55 |
| | Cytosolic | AdCa | 38 | 83 |
| | | SCC | 5 | 52 |

### Example 2

### Mitochondrial Hsp90 Regulation of Tumor Cell Motility

To begin investigating a role of mitochondrial Hsp90s in tumor cell movements, we used Gamitrinib (GA mitochondrial matrix inhibitor), a small molecule Hsp90 ATPase antagonist engineered to accumulate selectively in mitochondria (24). In these experiments, non-cytotoxic concentrations of Gamitrinib (23) suppressed the migration (FIG. 1A; FIG. 10A), and invasion (FIG. 1B; FIG. 10B) of tumor cell types. When tested in a more physiologic, 3-D model of cellular motility, Gamitrinib blocked tumor cell invasion in organotypic spheroids embedded in a collagen matrix (FIG. 1C), as evidence by nearly complete suppression of invasive length and invasive areas (FIG. 1D). In control experiments, Gamitrinib did not reduce tumor cell proliferation, compared to vehicle-treated cultures cells (FIG. 1E). Overall cell viability in a 3-D microenvironment was also not affected, by calcein-AM staining and 2-photon microscopy imaging of organotypic spheroids (FIG. 1F). Consistent with these findings, Gamitrinib also inhibited tumor cell migration in a wound closure assay at both 16 and 24 h time intervals (FIG. 1G). This effect was specific for the tumor cells, as normal human fibroblasts treated with a broad range of Gamitrinib concentrations did not affect migration in a wound closure assay at comparable time intervals (FIG. 1H).

As an independent approach, we next knocked down the expression of one of the targets of Gamitrinib in mitochondria, the Hsp90-like chaperone, Tumor Necrosis Factor Receptor-Associated Protein-1 (TRAP-1) (21). TRAP-1 silencing using pooled small interfering RNAs (siRNA) (FIG. 2A) reproduced the effect of Gamitrinib, and suppressed tumor cell migration (FIG. 2B). In contrast, TRAP-1 knockdown did not affect tumor cell proliferation, compared to cultures transfected with non-targeting siRNA (FIG. 2C). To validate the specificity of these findings, we next silenced TRAP-1 in tumor cell types using individual siRNA sequences (FIG. 2D). TRAP-1 knockdown under these conditions was also associated with significant inhibition of tumor cell migration (FIG. 2E), and invasion (FIG. 2F), whereas a control non-targeting siRNA was without effect.

### Example 3

### Cellular Requirements of Mitochondrial Hsp90 Control of Tumor Cell Motility

Consistent with inhibition of cell motility, exposure of tumor cells to Gamitrinib suppressed actin cytoskeletal assembly, with appearance of a rounded cell morphology, devoid of stress fibers and filopodia, by fluorescence microscopy (FIG. 11). Accordingly, Gamitrinib treatment resulted in nearly complete loss of cytoskeletal lamella dynamics as evidenced by single cell, time-lapse videomicroscopy analysis (FIG. 3A-C; FIG. 12A-12C), with profound inhibition of ruffling frequency and retraction speed in tumor cells (FIG. 3D). When analyzed for biochemical markers of cell motility, Gamitrinib-treated tumor cells exhibited loss of phosphorylation of Focal Adhesion Kinase (FAK) (25) on its auto-phosphorylation site, Tyr397 (26), and Src-phosphorylation site, Tyr925 (27) (FIG. 3E). Gamitrinib also inhibited the phosphorylation of other cell motility kinases, including Src on Tyr416 (FIG. 3E), and of group I p21-activated kinases, Pak-1,2 (28) on Ser199, Ser144 and Ser20 (FIG. 3F). The activation of Rho family small GTPases, Rac1 and Cdc42, which coordinate actin cytoskeleton reorganization, lamellipodia protrusion and directional cell movement (29), was also impaired by Gamitrinib treatment (FIG. 3G). Instead, Akt or ERK phosphorylation was not affected, and the total levels of these kinases remained unchanged (FIG. 3E).

We next asked whether modulation of signaling kinases participated in mitochondrial Hsp90s regulation of tumor cell motility. When transfected in tumor cells, recombinant FAK was phosphorylated, i.e. activated on Tyr397 and Tyr925 (FIG. 3H). Under these conditions, transfection of FAK nearly completely restored tumor cell migration and invasion in the presence of Gamitrinib (FIG. 3I). In contrast, transfected FAK had no effect on tumor cell proliferation in the presence or absence of Gamitrinib (FIG. 3J). Similarly, transfection of Src (FIG. 3K), or constitutively active Cdc42Val12 mutant (FIG. 3L) rescued the inhibition of tumor cell motility mediated by Gamitrinib (FIGS. 3K and L). Conversely, transfection of constitutively active RacVal12 did not reverse the effect of Gamitrinib on tumor cell invasion (FIG. 12D).

### Example 4

### Bioenergetics Requirement of Mitochondrial Hsp90S-Directed Tumor Cell Motility

In addition to opposing CypD-dependent permeability transition (21), mitochondrial Hsp90s maintain ATP production in tumor cells via retention of HK-II to the organelle outer membrane (23). To test whether this function in bioenergetics was important for tumor cell motility, we next treated tumor cells with the mitochondrial uncoupler carbonyl cyanide 3-chlorophenylhydrazone (CCCP), and looked for changes in cell motility. In these experiments, CCCP suppressed tumor cell migration (FIG. 13A, 13B), and invasion (FIG. 4A), and abolished FAK phosphorylation on Tyr397 and Tyr925 (FIG. 4B). Similar results were obtained in response to energy starvation induced by the non-hydrolyzable glucose analog, 2 deoxyglucose (2-DG), which also suppressed tumor cell migration (FIG. 13A, 13B), and invasion (FIG. 4A). Conversely, inhibition of Hsp90 chaperone activity in cytosol, but not mitochondria, with 17-allylamino demethoxygeldanamicyn (17-AAG), (24) did not affect tumor cell migration or invasion (FIG. 4A; FIG. 13A, 13B). As control for these experiments, treatment of tumor cells with CCCP, 2-DG or Gamitrinib did not affect cell proliferation (FIG. 4C), or cell death determined by Trypan blue exclusion (FIG. 13C), compared to vehicle-treated cultures.

To identify a potential link between mitochondrial bioenergetics and tumor cell motility, we next exposed tumor cells to progressively lower glucose concentrations, and looked at changes in metabolic markers. Consistent with current models of cellular responses to energy deprivation, glucose-starved LN229 cells exhibited increased phosphorylation of the energy sensor, AMPK, and concomitant activation of compensatory autophagy, as assessed by LC3 lipidation (FIG. 4D). Silencing of TRAP-1 by siRNA considerably exacerbated these compensatory responses to energy deprivation (FIG. 4D), demonstrating that TRAP-1 expression can dampen the activation of AMPK (23) and the induction of autophagy (30) during bioenergetics stress(es). Similar results were obtained with pharmacologic inhibition of mitochondrial Hsp90s with Gamitrinib, which also increased AMPK phosphorylation during nutrient deprivation, in a reaction partially reversed by addition of extracellular glucose (FIG. 4E). Consistent with a role of mitochondrial Hsp90s in bioenergetics, siRNA silencing of TRAP-1 reduced tumor cell production of ATP at limiting glucose concentrations, compared to control transfectants (FIG. 4F). To begin identifying the requirements of this response, we next cultivated tumor cells in the presence of increasing concentrations of galactose that is only used for oxidative phosphorylation. In the presence of glucose or 50% galactose, Gamitrinib did not significantly reduce tumor cell viability, compared to vehicle-treated cultures (FIG. 4G). However, when analyzed in 100% galactose concentrations, Gamitrinib induced nearly complete tumor cell death, whereas approximately 25% of cells in control cultures remained viable (FIG. 4G).

We next performed reciprocal experiments, and manipulated the expression of mitochondrial Hsp90s in normal NIH3T3 fibroblasts, which have low endogenous levels of these chaperones in mitochondria (21). Transfection of TRAP-1 in glucose-starving NIH3T3 cells attenuated phosphorylation of AMPK and of its substrate, acetyl-CoA carboxylase (ACC), compared to control transfectants (FIG. 4H). This was associated with increased ATP production at limiting or no glucose concentrations, as opposed to vector transfectants that had low levels of ATP production (FIG. 4I). When analyzed for cell motility, glucose- or amino acid-deprived NIH3T3 fibroblasts transfected with vector showed minimal invasion across Matrigel substrates (FIG. 4J). Conversely, expression of TRAP-1 was sufficient to promote invasion of nutrient-deprived NIH3T3 cells (FIG. 4J). In control experiments, recombinant TRAP-1 expression did not significantly affect overall mitochondrial function by an MTT assay (FIG. 14A), cell viability by Trypan blue exclusion (FIG. 14B) or cell proliferation by direct cell counting (FIG. 4K).

### Example 5

### Control of Tumor Cell Motility by AMPK-Autophagy Signaling

The data above have shown that AMPK phosphorylation correlates inversely with tumor cell movements (FIGS. 4H, J), and this response was further investigated. Silencing of AMPK using independent siRNA sequences (FIG. 5A, top) restored tumor cell motility in the presence of Gamitrinib (FIG. 5A, bottom), suggesting that AMPK activation was required for this response. siRNA knockdown of the upstream AMPK activator and tumor suppressor, liver kinase B1 (LKB1) (FIG. 5B, top) (14), produced the same effect, and rescued the inhibition of tumor cell invasion in the presence of Gamitrinib (FIG. 5B, bottom). We next asked whether a requirement by LKB1-AMPK signaling in this pathway involved de novo energy production. In these experiments, Gamitrinib inhibited ATP production in tumor cells in a concentration-dependent manner (FIG. 5C). In contrast, siRNA silencing of AMPK or LKB1 did not affect this response (FIG. 5C). Next, we performed the reciprocal experiments, and transfected tumor cells with vector or a constitutively active AMPK cDNA (AMPK^{CA}, FIG. 5D). Forced expression of active AMPK was sufficient to reproduce the effect of Gamitrinib, and inhibited tumor cell migration (FIG. 15A), and invasion (FIG. 5E). Similar to the data reported above for LKB1-AMPK signaling (FIG. 5C), constitutive expression of AMPK^{CA} was not associated with significant changes in ATP production in tumor cells (FIG. 5F). A control vector had no effect (FIGS. 5E, F).

Next, we looked downstream of LKB1-AMPK signaling (14), and asked whether activation of the mTORC1 pathway (15), and/or autophagy (31) contributed to tumor cell motility. Consistent with previous observations (15), inhibition of mTORC1 with rapamycin suppressed 4EBP1 phosphorylation in tumor cells (FIG. 5G). However, this had no effect on FAK phosphorylation (FIG. 5G), or tumor cell migration (FIG. 5H). Conversely, siRNA silencing of the essential autophagy gene, atg5 (FIG. 5I), partially rescued tumor cell invasion in the presence of Gamitrinib (FIG. 5J), indicating that activation of autophagy was required for mitochondrial Hsp90 regulation of tumor cell motility. To validate the role of autophagy in this pathway, we next silenced the expression of TRAP-1 together with AMPK or atg5 (FIG. 15B), and we looked at changes in tumor cell motility. Simultaneous siRNA knockdown of AMPK or atg5 restored tumor cell migration in the presence of TRAP-1 silencing by siRNA (FIG. 5K). In these experiments, simultaneous knockdown of the autophagy initiating UNC-51-like kinase (ULK1) (32) also rescued the inhibitory effect of TRAP-1 silencing on tumor cell motility (FIG. 5K).

### Example 6

### ULK1-FIP200 Regulation of Tumor Cell Motility

The data above suggest that the upstream autophagy regulator, ULK1 controlled tumor cell motility during bioenergetics stress, and this possibility was next investigated. Consistent with this model, expression of constitutively active AMPK^{CA} in tumor cells resulted in strong phosphorylation of ULK1 on Ser555, which has been implicated as a potential AMPK phosphorylation site (31, 32) (FIG. 6A). In contrast, transfection of AMPK^{CA} reduced ULK1 phosphorylation on Ser757, a potential mTORC1, (31) compared to vector control cells (FIG. 6A). Src phosphorylation on Tyr416 was also attenuated by expression of constitutively active AMPK, whereas the total levels of the kinases were not affected (FIG. 6A). We next asked whether energy starvation induced by Gamitrinib produced similar changes in ULK1 phosphorylation status. In these experiments, Gamitrinib induced early phosphorylation of AMPK and of its downstream substrate, ACC (FIG. 6B), in agreement with recent observations (23). This was associated with significant changes in the ratio of AMPK (Ser555)/mTORC1 (Ser757) phosphorylation, with rapid, i.e. within 1-2 h treatment, phosphorylation of ULK1 on Ser555, and decreased phosphorylation on Ser757 (FIG. 6B). Consistent with a model of ULK1 activation under these conditions, Gamitrinib also induced strong phosphorylation of Raptor (33) on its inhibitory site, Ser792, whereas the total level of ULK1 was not affected (FIG. 6B). As control, treatment of tumor cells with the AMPK activator, metformin produced similar results, with activation of AMPK and ACC, increased ULK1 phosphorylation on Ser555, and inhibition of mTORC1 signaling due to phosphorylation of Raptor on Ser792, and concomitant decreased phosphorylation of ULK1 on Ser757 (FIG. 6B).

We next asked whether these responses were specific to modulation of mitochondrial bioenergetics, and we treated tumor cells with 17-AAG, which inhibits Hsp90 chaperone activity in the cytosol, but not mitochondria. At variance with Gamitrinib, 17-AAG did not affect AMPK or ACC phosphorylation in tumor cells (FIG. 6C). Also, the ratio of ULK1 phosphorylation on the AMPK (Ser555)/mTORC1 (Ser757) site was unaffected, as 17-AAG equally reduced ULK1 phosphorylation on Ser757 and Ser555, and Raptor remained unphosphorylated on Ser792 (FIG. 6C). Although at the time points of this experiment, total ULK1 levels were not affected, a longer, 24-h exposure of tumor cells to 17-AAG caused ULK1 degradation (not shown), in agreement with recent observations (34).

We next silenced the expression of ULK1 by siRNA (FIG. 15C), and looked at biochemical and functional markers of cell motility in the presence or absence of Gamitrinib. ULK1 knockdown in tumor cells was sufficient to re-activate phosphorylation of FAK on Ty397 and Tyr925, and of Src on Tyr416 despite the presence of Gamitrinib (FIG. 6D). Functionally, this was associated with rescue of tumor cell migration (FIG. 6E, left) and invasion (FIG. 6E, right) from the inhibitory effect of Gamitrinib. To confirm that this response was specific, we next used ULK1-silenced tumor cells reconstituted with siRNA-insensitive vectors. Expression of WT ULK1 in these cells (FIG. 6F, top) restored the inhibition of tumor cell motility mediated by Gamitrinib (FIG. 6F, bottom). In contrast, expression of non-AMPK phosphorylatable ULK1 mutant (4SA), or a kinase-inactive (KI) ULK1 mutant (FIG. 6F, top) did not rescue tumor cell motility in the presence of Gamitrinib (FIG. 6F, bottom). In control, reconstitution studies with the various siRNA-insensitive ULK1 cDNAs did not significantly affect tumor cell proliferation (FIG. 6G).

In addition to atg13, ULK1 forms a complex with FIP200 (35), a molecule first identified as an endogenous inhibitor of FAK (36), and more recently implicated in autophagy (37), especially autophagosome formation (38, 39). Therefore, we next asked whether FIP200 functioned downstream of AMPK-ULK1 to control tumor cell motility. Consistent with this possibility, transfection of a FIP200 cDNA (FIG. 7A, top) abolished tumor cell migration (FIG. 15D), and invasion (FIG. 7A, bottom) compared to control transfectants. In contrast, tumor cell proliferation, was not affected (FIG. 7B). In reciprocal experiments, we silenced the expression of FIP200 using siRNA pools (FIG. 16A), or individual siRNA sequences (FIG. 7C, top) and looked at tumor cell motility. In these studies, FIP200 knockdown reversed the inhibitory effect of Gamitrinib on tumor cell migration and invasion (FIG. 16B and FIG. 16C, bottom), and restored the activating phosphorylation of FAK and Pak1, 2 (Ser144), compared with control siRNA transfectants (FIG. 7D). The regulation of FIP200 function has not been completely elucidated, but may involve a ULK1-dependent phosphorylation step. Consistent with this model, FIP200 analyzed from Gamitrinib-treated cells exhibited gel mobility retardation, suggestive of increased phosphorylation (FIG. 7E, left), and, accordingly, reacted with an antibody to pan-phosphorylated Ser (FIG. 7E). right). Finally, siRNA silencing of FIP200 (FIG. 7F) completely rescued the tumor cell migration in TRAP-1-depleted cells (FIG. 7G), mimicking the results obtained with Gamitrinib. In control experiments, FIP200 or TRAP-1 silencing, alone or in combination, did not affect tumor cell proliferation (FIG. 7H).

### Example 7

### Mitochondrial Hsp90s Control Metastasis, In Vivo

We next examined the impact of mitochondrial Hsp90s regulation of tumor cell motility in disease settings. First, we used a model of bone metastasis in which we silenced the expression of TRAP-1 in breast adenocarcinoma MDA-231 cells labeled with GFP (FIG. 2A), and injected them in the left ventricle of immunocompromised mice to look at their dissemination to the knee joint after 72 h. GFP+ cells transfected with non-targeting siRNA homed to the bone of injected animals as efficiently as non-transfected cultures (FIGS. 8A, B). In contrast, siRNA silencing of TRAP-1 suppressed tumor cell localization to bone (FIGS. 8A, B). In a second model of metastasis, we targeted the AMPK-ULK1 pathway, and we transfected lung adenocarcinoma H460 cells with ULK1 or constitutively active AMPK^{CA} cDNA. Injection of cells transfected with control vector into the spleen of immunocompromised mice gave rise to extensive metastatic foci in the liver within 11d (FIGS. 8C, D). In contrast, transfection of AMPK^{CA} or ULK1 cDNA reduced both the number and surface area of liver metastasis in reconstituted animals (FIGS. 8C, D).

Based on these results, we next asked whether AMPK-ULK1 regulation of cell motility was important in human tumors, and we studied a series of non-small cell lung cancer (NSCLC) patients with available clinical outcome data (see Table 1). ULK1 expression was elevated in NSCLC, especially adenocarcinoma (AdCa) histotypes, compared to squamous cell carcinoma (SCC) (FIG. 17A), but did not correlate with overall survival (FIG. 17B). Immunoreactivity for phosphorylated ULK1 on Ser555 (FIG. 8E, F) or Ser757 (FIGS. 8E, G) was also increased in NSCLC patients, compared to normal lung, predominantly in AdCa (FIGS. 8F, G). When stratified for disease outcome, phosphorylation of ULK1 on Ser757 was associated with shortened overall survival (FIG. 8H; FIG. 18C; Table 2). This trend was mostly observed in patients with AdCa histotype (FIG. 17D), independently of tumor size or lymph node involvement (FIG. 17E). In these cases, preferential activation of the mTORC1 (Ser757) over AMPK (Ser555) pathway in ULK1 (31, 32) correlated with larger tumor size (FIG. 17F; Table 2), and decreased overall survival (FIG. 8I).

### Conclusions from Examples 1-7

We have shown that tumor cells utilize mitochondrial Hsp90s-directed protein folding (21) to produce ATP (23) under conditions of stress, such as nutrient deprivation or amino acids shortage (12). In spite of nutrient starvation, this adaptive mechanism is sufficient to dampen the activation of the energy sensor and tumor suppressor, AMPK (14, 15), and limit the induction of another tumor suppression mechanism, autophagy (18) (FIG. 9). When analyzed in the context of cell movements, this pathway maintained cytoskeletal dynamics through persistent phosphorylation of multiple cell motility kinases, and released FAK (25, 26) from inhibition by an ULK1-FIP200 autophagy-initiating complex (38, 39) (FIG. 9). Functionally, this translated in enhanced tumor cell invasion, in vivo, metastatic dissemination to bone or liver in mouse models of disease, and shortened overall survival in patients with NSCLC (FIG. 9).

The crosstalk between bioenergetics stress imposed by nutrient deprivation, activation of AMPK and induction of autophagy in the dynamics of tumor growth is complex, and likely carries different functional implications depending on disease stage and cellular context. Accordingly, these mechanisms have been variously linked to tumor suppression (40), or, conversely, cell survival under metabolic stress (41), or tumor adaptation (18). Our results show that AMPK activation, while potentially important for cell survival (41), also provides a strong barrier against tumor cell motility and metastasis, in a pathway reversed by mTORC 1 activation (15). This model is in line with developmental roles of AMPK (42), and LKB1 (43) in epithelial polarity and cytoskeletal remodeling during metabolic stress (44), and anti-metastatic properties proposed for LKB1 (45).

A key effector of tumor cell motility under nutrient deprivation downstream of AMPK was identified here as the ULK1-FIP200 complex (32). Together with atg13, this multiprotein interactor functions as an upstream initiator of autophagy (37, 38), potentially coordinating the process of autophagosome formation (38). There is evidence for a role of ULK1-mediated phosphorylation (39) in the regulation of FIP200, consistent with our findings that ULK1 kinase activity is required for tumor cell motility, and that FIP200 exhibits phosphorylation-dependent gel retardation in energy-impaired tumor cells. In parallel with its role in autophagy (38), activated FIP200 also functions as an endogenous inhibitor of FAK (37), shutting off the multifunctional properties of this kinase in cell motility, invasion, proliferation and survival (25) that are often exploited in disparate cancers (26).

Mitochondrial Hsp90-directed bioenergetics emerged here as an adaptive mechanism that overcomes this global tumor suppressive network and enables FAK-dependent tumor cell invasion in face of nutrient deprivation. Mechanistically, this pathway involves retention of the first enzyme of the glycolytic cascade, HK-II, to the mitochondrial outer membrane (4) via regulation of CypD folding (23). However, other mechanisms of energy production are also plausible, and galactose challenge experiments presented here pointed to a role of mitochondrial Hsp90s in maintaining a residual level of oxidative phosphorylation, consistent with a role of these molecules in oxygen consumption (23).

The pathogenetic context for these observations (FIG. 9) resides in the highly unfavorable conditions of tumor growth, in vivo, chronically depleted of oxygen and nutrients, and constantly exposed to oxidative stresses (12). Despite these challenges, a nutrient-impaired microenvironment has been causally linked to tumor progression (1), and, accordingly, mitochondrial Hsp90-directed bioenergetics under energy deprivation enabled metastatic dissemination, in vivo. Conversely, loss of TRAP-1 expression in mitochondria or gain of activity by the autophagy regulators, AMPK or ULK1 antagonized tumor cell motility, and suppressed metastatic dissemination in mice. Despite the ambivalent nature of autophagy in tumor progression (17), there is now considerable effort to target the regulators of this pathway for cancer therapy. Our data highlight the complexity of this approach, and suggest that disabling autophagy-initiating molecules, in particular ULK1, may have detrimental contraindications, not only by removing autophagy-associated tumor cell death (17), but also by unrestraining FAK from FIP200 inhibition (36), promoting paradoxical tumor cell invasion and metastasis.

As a pivotal effector of this response, there is evidence that ULK1 regulation is achieved by differential phosphorylation, but controversy exists as to the reciprocal roles of AMPK or mTORC1 kinases in this process (35), and whether these modifications have disease relevance, in vivo. The data here suggest that a relative ratio of ULK1 phosphorylation on putative AMPK (Ser555) (31, 32) or mTORC1 (Ser757) (31) site(s) may better predict downstream responses of autophagy, and, consequently, tumor cell motility and invasion. This approach may have clinical utility, as we observed that preferential ULK1 phosphorylation by mTORC1 over AMPK correlates with disease progression and shortened overall survival in NSCLC patients. These data are consistent with a role of mTORC1 signaling (15) as a disease driver and potential therapeutic target in lung cancer (46), and suggest that AMPK activation in established malignancies, in vivo, may continue to provide as a tumor suppressor function, possibly linked to activation of autophagy (14) and inhibition of metastasis (this study).

In sum, mitochondrial Hsp90-directed tumor cell metabolism (23) functions as a pivotal mediator of tumor cell motility and invasion when nutrients are scarce, consistent with the nearly ubiquitous over-expression of these chaperones in advanced disease, in vivo (21). Although there has been considerable progress in mapping the transcriptional requirements of metastasis (47), and the cellular (48), and genetic (49, 50) aspects of this process have come into better focus, disseminated tumors are incurable, carrying considerable morbidity and mortality. Instead, subcellular targeting of mitochondrial Hsp90s as upstream regulators of tumor bioenergetics-cell invasion signaling (FIG. 9) is feasible (30), and may simultaneously disable key signaling nodes of tumor energy production (23), and cell survival (21), indispensable for metastatic competency, in vivo.

The present invention is based upon the determination that interference with Hsp90-directed protein folding in mitochondria triggers cellular starvation, with decreased ATP production and activation of the energy sensor LKB1-AMPK kinase axis (i). In turn, active AMPK phosphorylates its downstream target and autophagy initiator, ULK1 (ii). Third, activated ULK1 maintains focal adhesion kinase (FAK) under inhibition by the autophagy regulator, FIP200, thus suppressing tumor cell motility (iii). This places changes in the ATP/AMP ratio as the pivotal upstream requirement of this response, and the activation of the ULK1 autophagy regulator as the downstream effector that blunts FAK-dependent cell motility. The data presented in the examples demonstrates this mechanism. Accordingly, there is no loss of cell viability at any stage of this pathway (see, e.g., FIGS. 1E, 2C, 3J, 4C and K, 6G, and 7B and H). The roles of active LKB1 and AMPK in this response do not encompass changes in ATP production (see, e.g., FIGS. 5C and 5F). ULK1 activation of FIP200 involves Ser phosphorylation (see, FIG. 7E). The data also demonstrate that mitochondrial Hsp90-directed bioenergetics contributes to residual oxidative phosphorylation in tumor cells as determined in galactose challenge experiments (see e.g. FIG. 4G). FIG. 9 presents a model that summarizes the pathway.

The inventors demonstrate that targeting mitochondrial Hsp90s with Gamitrinib (see, e.g., FIGS. 1E, 3J, 4C, 6G and 7B), or siRNA silencing (see, e.g., FIGS. 2C and 7H), does not affect tumor cell proliferation. The data further demonstrate that AMPK, ULK1 and atg5 are required for inhibition of cell motility induced by siRNA silencing of TRAP-1, thus mirroring the data obtained with Gamitrinib. These results are shown in FIG. 5K. The ability of Gamitrinibs to inhibit the ATPase catalytic activity of mitochondrial Hsp90s has been reported (24), which demonstrated that geldanamycin/17-AAG- as well as non-geldanamycin-based Hsp90 inhibitors fail to accumulate in mitochondria, and, accordingly, do not induce acute mitochondrial dysfunction.

17-AAG causes ULK1 degradation, as expected from a bona fide Hsp90 client protein. Consistent with the method presented here (see FIG. 9), ULK1 degradation by 17-AAG is expected to release FAK from FIP200 inhibition and thus maintain tumor cell motility, which is what is observed experimentally in FIG. 4A. The effect of 17-AAG on biochemical markers of aULK1 activation were tested side-by-side with Gamitrinib. These data are shown in a FIG. 6C, and demonstrate that, differently from Gamitrinib, 17-AAG does not modulate AMPK or ACC phosphorylation, attenuates ULK1 phosphorylation on both the AMPK (Ser555) and mTORC1 (Ser757) sites, and does not inhibit mTORC1 signaling (Raptor phosphorylation). These data reinforce the specificity of the pathway and the uniqueness of targeting Hsp90s in mitochondria.

The contribution of mitochondrial Hsp90s to oxidative phosphorylation in galactose challenge experiments has been investigated and the results presented in FIG. 4G. These data demonstrate that inhibition of mitochondrial Hsp90s with Gamitrinib completely abrogates cellular viability in the presence of galactose. In contrast, control cells retained ˜25% viability under conditions of 100% galactose (FIG. 4G). These data suggest that, in addition to retaining HK-II to the organelle's outer membrane (23), mitochondrial Hsp90s contribute to energy production via residual (20-25%) oxidative phosphorylation. This conclusion is also consistent with previous data that Gamitrinib inhibited O₂ consumption in tumor cells (23). The concentrations of glutamine present in the growth medium have now been clarified throughout the revised manuscript.

Although there has been evidence in the literature that targeting modulators of cellular bioenergetics, for instance, HK-II, attenuates tumor cell migration, this pathway was proposed to involve inadequate ATP production to support cellular movements. Instead, our data supports that loss of bioenergetics (as induced by targeting mitochondrial Hsp90s) triggers cellular starvation, activates autophagy, and ultimately shuts down tumor cell motility via FIP200, independently of ATP production. This method also differs from other data that in the past linked tumor bioenergetics to cell motility, implicating increased lactate production through aerobic glycolysis as a biochemical mediator of enhanced tumor cell migration. The data highlight the complexity and potential contra-indications of targeting autophagy for cancer therapeutics, a strategy that would result in unrestrained FAK signaling and paradoxical increased tumor cell invasion. Taken together, the method described herein provides a novel cellular context for the interplay between tumor bioenergetics, autophagy, and cell motility. Mechanistically, this hinges on the adaptive function of Hsp90s in mitochondria, and the emerging ability of these molecules to regulate both aerobic glycolysis through HK-II retention to the organelle's outer membrane (23), and residual oxidative phosphorylation as demonstrated herein.

FIG. 3H shows that FAK over-expressed in tumor cells is highly phosphorylated on Tyr397 and Tyr9252.

Potential changes in tumor cell proliferation or apoptosis have been examined after treatment with 2-DG or CCCP. The results are shown in FIGS. 4C and 13C, and demonstrate that exposure to 2-DG or CCCP does not affect cell proliferation, by direct cell counting (FIG. 4C) or cell death, by Trypan blue exclusion (FIG. 13C). We have also examined a potential non-specific toxicity of amino acid and glucose deprivation. As shown in FIG. 4K and FIG. 14B, these treatments do not affect cell proliferation (FIG. 4K), or cell death (FIG. 14B).

Gamitrinib increases AMPK phosphorylation in a reaction reversed by addition of exogenous glucose (FIG. 4E), and inhibits ATP production in a dose-dependent manner (FIG. 5C). This is consistent with previous data that Gamitrinib suppresses ATP production, stimulates AMPK phosphorylation and inhibits mTORC1 signaling in tumor cells (23).

siRNA knockdown of AMPK or LKB 1 does not rescue ATP production in cells treated with Gamitrinib (FIG. 5C). Conversely, silencing of AMPK (FIG. 5A) or LKB1 (FIG. 5B) fully rescues the inhibition of tumor cell motility mediated by Gamitrinib. We also show that expression of constitutively active AMPK (AMPK^{CA}) does not affect ATP production in two different tumor cell types (FIG. 5F), consistent with the model that AMPK and LKB 1 act downstream of ATP imbalance, and modulate tumor cell motility via autophagy activation, i.e. ULK1. Plasmid over-expression of TRAP-1 in non-transformed NIH3T3 cells elevates ATP levels (FIG. 4I), and dampens AMPK activation in low glucose concentrations (FIG. 4H).

Gamitrinib increases Ser phosphorylation of FIP200, in vivo (FIG. 7E) consistent with a model of ULK1 phosphorylation of FIP200.

Expression of non-AMPK phosphorylatable ULK1 did not fully restore cell motility in the presence of Gamitrinib (FIG. 6F). Although this response was still highly statistically significant, it is possible that other phosphorylation events of ULK1 participate in this regulation.

The statistical analysis throughout the experiments was carried out on repeats of individual experiments, each with multiple replicates. Additional experiments were also repeated to achieve n=3 of selected experiments. The aggregate data are now shown in FIGS. 5A, 5B, 6F and 7G.

A graphic model of the proposed pathway connecting tumor bioenergetics, activation of autophagy and modulation of tumor cell motility is presented as FIG. 9.

Gamitrinib has been previously report to inhibit bone metastatic disease in both orthotopic xenograft models (58), as well as genetic mouse models (59).

Here, we used a more molecular approach of siRNA silencing or plasmid over-expression to dissect the molecular requirements of the Gamitrinib target, TRAP-1 (FIG. 7A, 7B), and its new regulators, AMPK and ULK1 (FIG. 7C, 7D) in this pathway. Here, we show that tumors maintain energy production under nutrient deprivation through the function of Heat Shock Protein-90 (Hsp90)¹⁰ chaperones in mitochondria¹¹. In turn, this attenuates AMPK activation, preserves cytoskeletal dynamics⁹, and promotes metastasis by releasing Focal Adhesion Kinase (FAK)¹² from inhibition by the autophagy regulator, FIP200^{13,14}. Thus, mitochondrial Hsp90-directed bioenergetics promotes tumor cell invasion under energetic stress, and provides a tractable target for anti-metastatic therapies. See, Caino et al, Metabolic stress regulates cytoskeletal dynamics and metastasis of cancer cells, J Clin Invest. 2013; 123(7):2907-2920, including supplemental materials, which is specifically incorporated herein by reference in its entirety.

### Example 8

### Additional Data

In a model of patient-derived and treatment-naïve GBM organotypic cultures, it was found that treatment with a PI3K inhibitor increased the expression of genes associated with growth factor signaling (EGF, ERK/MAPK, VEGF, ErbB, PDGF), metabolic sensing (AMPK, insulin and glucocorticoid receptors), cytoskeletal remodeling (Rho, Rac), and cell movement (FAK, HGF). These transcriptional changes clustered into two main gene networks of resistance to cell death and increased cell motility. In both GBM organoids and LN229 cells, this was associated with phosphorylation of several growth factor receptors (EGFR and related members, Insulin-R, IGF1-R, FGFR-2α and PDGFR), as well as kinases (RYK, ALK, DDR1, Ax1 and Ephrin) implicated in cell movement. Regulators of “stemness”, epithelial-mesenchymal transition, and cell motility (KLF4, NUMB, CD44, NANOG, and HGF) were also transcriptionally modulated by PI3K inhibition.

We next asked whether these bioenergetics and transcriptional changes caused a new tumor phenotype. Following treatment with a PI3K inhibitor, tumor cells acquired markers of senescence with increased β-galactosidase staining, and higher PML nuclear body number (data not shown). They also became quiescent, arrested in the G1 phase of the cell cycle, and had reduced proliferation, without significant decrease in viability. Instead, PI3K inhibition triggered considerably enhanced tumor cell invasion across Matrigel-coated Transwell membranes, as well as in 3D spheroids embedded in a collagen matrix. This was associated with higher phosphorylation of several cell motility kinases, and this was relevant because silencing of FAK or Src by small interfering RNA reduced tumor cell invasion induced by PI3K inhibition. We also observed that knockdown of Akt isoforms had the same effect, and abolished the increased tumor cell invasion.

Technical and scientific terms used throughout this specification have the same meaning as commonly understood by one of ordinary skill in the art to which this invention belongs and by reference to published texts, which provide one skilled in the art with a general guide to many of the terms used in the present application. Any definitions provided herein are provided for clarity only and are not intended to limit the claimed invention. As used herein, the terms “a” or “an”, refers to one or more, for example, “a cell marker” is understood to represent one or more cell markers. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

Each patent, patent application, and publication, including publications listed herein and publically available nucleic acid, peptide sequence or small molecule structure cited throughout the disclosure, are expressly incorporated herein by reference in its entirety. Embodiments and variations of this invention other than those specifically disclosed above may be devised by others skilled in the art without departing from the true spirit and scope of the invention. The appended claims include such embodiments and equivalent variations.

### DOCUMENTS

1. Kroemer, G., and Pouyssegur, J. 2008. Tumor Cell Metabolism: Cancer's Achilles' Heel. Cancer Cell 13:472-482.
2. Buchakjian, M. R., and Kornbluth, S. 2010. The engine driving the ship: metabolic steering of cell proliferation and death. Nat Rev Mol Cell Biol 11:715-727.
3. Koppenol, W. H., Bounds, P. L., and Dang, C. V. 2011. Otto Warburg's contributions to current concepts of cancer metabolism. Nat Rev Cancer 11:325-337.
4. Vander Heiden, M. G., Cantley, L. C., and Thompson, C. B. 2009. Understanding the Warburg effect: the metabolic requirements of cell proliferation. Science 324:1029-1033.
5. Gordan, J. D., Lal, P., Dondeti, V. R., Letrero, R., Parekh, K. N., Oquendo, C. E., Greenberg, R. A., Flaherty, K. T., Rathmell, W. K., Keith, B., et al. 2008. HIF-alpha effects on c-Myc distinguish two subtypes of sporadic VHL-deficient clear cell renal carcinoma. Cancer Cell 14:435-446.
6. Dang, L., White, D. W., Gross, S., Bennett, B. D., Bittinger, M. A., Driggers, E. M., Fantin, V. R., Jang, H. G., Jin, S., Keenan, M. C., et al. 2009. Cancer-associated IDH1 mutations produce 2-hydroxyglutarate. Nature 462:739-744.
7. Zhao, S., Lin, Y., Xu, W., Jiang, W., Zha, Z., Wang, P., Yu, W., Li, Z., Gong, L., Peng, Y., et al. 2009. Glioma-derived mutations in IDH1 dominantly inhibit IDH1 catalytic activity and induce HIF-1alpha. Science 324:261-265.
8. Koivunen, P., Lee, S., Duncan, C. G., Lopez, G., Lu, G., Ramkissoon, S., Losman, J. A., Joensuu, P., Bergmann, U., Gross, S., et al. 2012. Transformation by the (R)-enantiomer of 2-hydroxyglutarate linked to EGLN activation. Nature 483:484-488.
9. Lu, C., Ward, P. S., Kapoor, G. S., Rohle, D., Turcan, S., Abdel-Wahab, O., Edwards, C. R., Khanin, R., Figueroa, M. E., Melnick, A., et al. 2012. IDH mutation impairs histone demethylation and results in a block to cell differentiation. Nature 483:474-478.
10. Wellen, K. E., and Thompson, C. B. 2012. A two-way street: reciprocal regulation of metabolism and signalling. Nat Rev Mol Cell Biol 13:270-276.
11. Schafer, Z. T., Grassian, A. R., Song, L., Jiang, Z., Gerhart-Hines, Z., Irie, H. Y., Gao, S., Puigserver, P., and Brugge, J. S. 2009. Antioxidant and oncogene rescue of metabolic defects caused by loss of matrix attachment. Nature 461:109-113.
12. Laderoute, K. R., Amin, K., Calaoagan, J. M., Knapp, M., Le, T., Orduna, J., Foretz, M., and Viollet, B. 2006. 5′-AMP-activated protein kinase (AMPK) is induced by low-oxygen and glucose deprivation conditions found in solid-tumor microenvironments. Mol Cell Biol 26:5336-5347.
13. Yoo, L. I., Chung, D. C., and Yuan, J. 2002. LKB1—a master tumour suppressor of the small intestine and beyond. Nat Rev Cancer 2:529-535.
14. Mihaylova, M. M., and Shaw, R. J. 2011. The AMPK signalling pathway coordinates cell growth, autophagy and metabolism. Nat Cell Biol 13:1016-1023.
15. Wullschleger, S., Loewith, R., and Hall, M. N. 2006. TOR signaling in growth and metabolism. Cell 124:471-484.
16. Behrends, C., Sowa, M. E., Gygi, S. P., and Harper, J. W. 2010. Network organization of the human autophagy system. Nature 466:68-76.
17. Kimmelman, A. C. 2011. The dynamic nature of autophagy in cancer. Genes Dev. 25:1999-2010.
18. White, E. 2012. Deconvoluting the context-dependent role for autophagy in cancer. Nat Rev Cancer.
19. Friedl, P., and Alexander, S. 2011. Cancer invasion and the microenvironment: plasticity and reciprocity. Cell 147:992-1009.
20. Taipale, M., Jarosz, D. F., and Lindquist, S. 2010. Hsp90 at the hub of protein homeostasis: emerging mechanistic insights. Nat Rev Mol Cell Biol 11:515-528.
21. Kang, B. H., Plescia, J., Dohi, T., Rosa, J., Doxsey, S. J., and Alfieri, D. C. 2007. Regulation of tumor cell mitochondrial homeostasis by an organelle-specific Hsp90 chaperone network. Cell 131:257-270.
22. Green, D. R., and Kroemer, G. 2004. The pathophysiology of mitochondrial cell death. Science 305:626-629.
23. Chae, Y. C., Caino, M. C., Lisanti, S., Ghosh, J. C., Dohi, T., Danial, N. N., Villanueva, J., Ferrero, S., Vaira, V., Santambrogio, L., et al. 2012. Control of tumor bioenergetics and survival stress signaling by mitochondrial HSP90s. Cancer Cell 22:331-344.
24. Kang, B. H., Plescia, J., Song, H. Y., Meli, M., Colombo, G., Beebe, K., Scroggins, B., Neckers, L., and Alfieri, D. C. 2009. Combinatorial drug design targeting multiple cancer signaling networks controlled by mitochondrial Hsp90. J Clin Invest 119:454-464.
25. Frame, M. C., Patel, H., Serrels, B., Lietha, D., and Eck, M. J. 2010. The FERM domain: organizing the structure and function of FAK. Nat. Rev. Mol. Cell. Biol. 11:802-814.
26. Schaller, M. D. 2010. Cellular functions of FAK kinases: insight into molecular mechanisms and novel functions. J Cell Sci 123:1007-1013.
27. Deramaudt, T. B., Dujardin, D., Hamadi, A., Noulet, F., Kolli, K., De Mey, J., Takeda, K., and Ronde, P. 2011. FAK phosphorylation at Tyr-925 regulates cross-talk between focal adhesion turnover and cell protrusion. Mol Biol Cell 22:964-975.
28. Kumar, R., Gururaj, A. E., and Barnes, C. J. 2006. p21-activated kinases in cancer. Nat. Rev. Cancer 6:459-471.
29. Raftopoulou, M., and Hall, A. 2004. Cell migration: Rho GTPases lead the way. Dev. Biol. 265:23-32.
30. Siegelin, M. D., Dohi, T., Raskett, C. M., Orlowski, G. M., Powers, C. M., Gilbert, C. A., Ross, A. H., Plescia, J., and Alfieri, D. C. 2011. Exploiting the mitochondrial unfolded protein response for cancer therapy in mice and human cells. J Clin Invest 121:1349-1360.
31. Kim, J., Kundu, M., Viollet, B., and Guan, K.-L. 2011. AMPK and mTOR regulate autophagy through direct phosphorylation of Ulk1. Nat. Cell Biol 13:132-141.
32. Egan, D. F., Shackelford, D. B., Mihaylova, M. M., Gelino, S., Kohnz, R. A., Mair, W., Vasquez, D. S., Joshi, A., Gwinn, D. M., Taylor, R., et al. 2011. Phosphorylation of ULK1 (hATG1) by AMP-activated protein kinase connects energy sensing to mitophagy. Science 331:456-461.
33. Kim, D H, Sarbassov, D. D., Ali, S. M., King, J. E., Latek, R. R., Erdjument-Bromage, H., Tempst, P., and Sabatini, D. M. 2002. mTOR interacts with raptor to form a nutrient-sensitive complex that signals to the cell growth machinery. Cell 110:163-175.
34. Joo, J. H., Dorsey, F. C., Joshi, A., Hennessy-Walters, K. M., Rose, K. L., McCastlain, K., Zhang, J., Iyengar, R., Jung, C. H., Suen, D. F., et al. 2011. Hsp90-Cdc37 chaperone complex regulates Ulk1- and Atg13-mediated mitophagy. Mol Cell 43:572-585.
35. Alers, S., Loffler, A. S., Wesselborg, S., and Stork, B. 2012. Role of AMPK-mTOR-Ulk1/2 in the regulation of autophagy: cross talk, shortcuts, and feedbacks. Mol Cell Biol 32:2-11.
36. Abbi, S., Ueda, H., Zheng, C., Cooper, L. A., Zhao, J., Christopher, R., and Guan, J. L. 2002. Regulation of focal adhesion kinase by a novel protein inhibitor FIP200. Mol Biol Cell 13:3178-3191.
37. Wei, H., Wei, S., Gan, B., Peng, X., Zou, W., and Guan, J. L. 2011. Suppression of autophagy by FIP200 deletion inhibits mammary tumorigenesis. Genes Dev 25:1510-1527.
38. Hara, T., Takamura, A., Kishi, C., Iemura, S., Natsume, T., Guan, J. L., and Mizushima, N. 2008. FIP200, a ULK-interacting protein, is required for autophagosome formation in mammalian cells. J Cell Biol 181:497-510.
39. Jung, C. H., Jun, C. B., Ro, S. H., Kim, Y. M., Otto, N. M., Cao, J., Kundu, M., and Kim, D. H. 2009. ULK-Atg13-FIP200 complexes mediate mTOR signaling to the autophagy machinery. Mol Biol Cell 20:1992-2003.
40. van Veelen, W., Korsse, S. E., van de Laar, L., and Peppelenbosch, M. P. 2011. The long and winding road to rational treatment of cancer associated with LKB1/AMPK/TSC/mTORC1 signaling. Oncogene 30:2289-2303.
41. Jeon, S.-M., Chandel, N. S., and Hay, N. 2012. AMPK regulates NADPH homeostasis to promote tumour cell survival during energy stress. Nature 485:661-665.
42. Lee, J. H., Koh, H., Kim, M., Kim, Y, Lee, S. Y., Karess, R. E., Lee, S. H., Shong, M., Kim, J M, Kim, J, et al. 2007. Energy-dependent regulation of cell structure by AMP-activated protein kinase. Nature 447:1017-1020.
43. Martin, S. G., and St Johnston, D. 2003. A role for Drosophila LKB1 in anterior-posterior axis formation and epithelial polarity. Nature 421:379-384.
44. Mirouse, V., Swick, L. L., Kazgan, N., St Johnston, D., and Brenman, J. E. 2007. LKB1 and AMPK maintain epithelial cell polarity under energetic stress. J Cell Biol 177:387-392.
45. Ji, H., Ramsey, M. R., Hayes, D. N., Fan, C., McNamara, K., Kozlowski, P., Torrice, C., Wu, M. C., Shimamura, T., Perera, S. A., et al. 2007. LKB1 modulates lung cancer differentiation and metastasis. Nature 448:807-810.
46. Khokhar, N. Z., Altman, J. K., and Platanias, L. C. 2011. Emerging roles for mammalian target of rapamycin inhibitors in the treatment of solid tumors and hematological malignancies. Curr Opin Oncol 23:578-586.
47. Bos, P. D., Zhang, X. H., Nadal, C., Shu, W., Gomis, R. R., Nguyen, D. X., Minn, A. J., van de Vijver, M. J., Gerald, W. L., Foekens, J. A., et al. 2009. Genes that mediate breast cancer metastasis to the brain. Nature 459:1005-1009.
48. Valastyan, S., and Weinberg, R. A. 2011. Tumor metastasis: molecular insights and evolving paradigms. Cell 147:275-292.
49. Lujambio, A., and Lowe, S. W. 2012. The microcosmos of cancer. Nature 482:347-355.
50. Scott, K. L., Nogueira, C., Heffernan, T. P., van Doom, R., Dhakal, S., Hanna, J. A., Min, C., Jaskelioff, M., Xiao, Y., Wu, C. J., et al. 2011. Proinvasion metastasis drivers in early-stage melanoma are oncogenes. Cancer Cell 20:92-103.
51. Jamieson-Gladney, W. L., Zhang, Y., Fong, A. M., Meucci, O., and Fatatis, A. 2011. The chemokine receptor CX(3)CR1 is directly involved in the arrest of breast cancer cells to the skeleton. Breast Cancer Res 13:R91.
52. Russell, M. R., Jamieson, W. L., Dolloff, N. G., and Fatatis, A. 2009. The alpha-receptor for platelet-derived growth factor as a target for antibody-mediated inhibition of skeletal metastases from prostate cancer cells. Oncogene 28:412-421.
53. Mehrotra, S., Languino, L. R., Raskett, C. M., Mercurio, A. M., Dohi, T., and Altieri, D. C. 2010. TAP regulation of metastasis. Cancer Cell 17:53-64.
54. Flaiz, C., Chernoff, J., Ammoun, S., Peterson, J. R., and Hanemann, C. O. 2009. PAK kinase regulates Rac GTPase and is a potential target in human schwannomas. Exp. Neurol. 218:137-144.
55. Hinz, B., Alt, W., Johnen, C., Herzog, V., and Kaiser, H.-W. 1999. Quantifying Lamella Dynamics of Cultured Cells by SACED, a New Computer-Assisted Motion Analysis. Exp. Cell Res. 251:234-243.
56. Kinoshita, E., Kinoshita-Kikuta, E., and Koike, T. 2009. Separation and detection of large phosphoproteins using Phos-tag SDS-PAGE. Nat. Protocols 4:1513-1521.
57. Barberis, M., Pellegrini, C., Cannone, M., Arizzi, C., Coggi, G., and Bosari, S. 2008. Quantitative PCR and HER2 testing in breast cancer: a technical and cost-effectiveness analysis. Am. J. Clin. Pathol. 129:563-570.
58. Kang, B H et al, 2010. Preclinical Characterization of Mitochondria-Targeted Small Molecule Hsp90 Inhibitors, Gamitrinibs, in Advanced Prostate Cancer. Clin Cancer Res 16:4779-4788
59. Kang, B H et al, 2011. Targeted inhibition of mitochondrial Hsp90 suppresses localised and metastatic prostate cancer growth in a genetic mouse model of disease. Br J Cancer 104: 629-634.

1. A method for inhibiting tumor cell migration in a mammalian subject comprising:
(a) inhibiting, suppressing or down-regulating focal adhesion kinase (FAK) activity, expression, or phosphorylation of FAK in a cancer or tumor cell;
(b) inhibiting, suppressing or down-regulating ULK1 kinase activity, expression or phosphorylation of ULK1 on Ser757 in a cancer or tumor cell;
(c) inhibiting, suppressing or down-regulating activation, expression or signaling of the mTORC1 (Ser757) pathway in a cancer or tumor cell; or
(d) inhibiting, suppressing or down-regulating activity or expression of TRAP1 in a cancer or tumor cell;
(e) increasing, stimulating or activating AMPK activity or expression in a cancer or tumor cell;
(f) increasing, stimulating or activating LKB1 activity or expression in a cancer or tumor cell;
(g) increasing, stimulating or activating FIP200 activity, expression or phosphorylation in a cancer or tumor cell.

2. The method according to claim 1, further comprising administering to a subject a therapeutically effective dose of at least one of:
(a) a composition comprising a molecule that inhibits, suppresses or down-regulates focal adhesion kinase (FAK) activity, expression or phosphorylation of FAK in a cancer cell.
(b) a composition comprising a molecule that inhibits, suppresses or down-regulates ULK1 kinase activity or expression;
(c) a composition comprising a molecule that inhibits, suppresses or down-regulates activation, expression or signaling of the mTORC1 (Ser757) pathway;
(d) a compositions comprising a molecule that inhibits, suppresses or down-regulates activity or expression of TRAP1;
(e) a composition comprising a molecule that increases, stimulates or activates AMPK activity or expression;
(f) a composition comprising a molecule that increases, stimulates or activates LKB1 activity or expression; and
(g) a composition comprising a molecule that increases, stimulates or activates FIP200 activity or expression or phosphorylation.

3. The method according to claim 2, wherein said composition (b) suppresses phosphorylation of ULK1 on Ser757 in subjects with lung cancer.

4. The method according to claim 2, wherein the composition (c) is administered to subjects with lung cancer.

5. The method according to claim 2, wherein the compositions (c) and (e) adjust the ratio of the AMPK pathway to mTORC 1 pathway in favor of the AMPK pathway.

6. The method according to claim 1, wherein the cancer is a glioblastoma a prostate adenocarcinoma, a lung adenocarcinoma, non-small cell lung adenocarcinoma, squamous cell carcinoma, liver cancer, breast adenocarcinoma or melanoma.

7. The method according to claim 1, wherein the subject has an established malignancy.

8. The method according to claim 1, wherein the administration occurs at a selected time during the course of cancer therapy.

9. The method according to claim 1, wherein the administration is part of a protocol of neoadjuvant therapy.

10. The method according to claim 2, wherein the composition comprises Gamitrinib or an antibody to FAK, ULK1, mTORC1, or TRAP1.

11. The method according to claim 2, further comprising administering the suboptimal dose of the composition prior to or during a course of chemotherapy or radiation to reduce the size of an existing primary tumor.

12. The method according to claim 2, further comprising administering a suboptimal dose of the composition prior to or during surgery performed to debulk or remove a primary tumor.

13. The method according to claim 2, comprising administering the suboptimal dose of the composition after surgery performed to debulk or remove a primary tumor.

14. The method according to claim 2, further comprising administering the suboptimal dose of the composition prior to or during a second or repeated course of chemotherapy or radiation.

15. The method according to claim 14 wherein the second or repeated course is post-surgery.

16. The method according to claim 2, further comprising administering a continuous course of a suboptimal a dose of the composition to a patient from prior to a first round of pre-surgical chemotherapy, during and after surgery, and prior to and after a second round of post-surgical chemotherapy.

17. The method according to claim 2, further comprising administering periodic suboptimal doses of the composition to a patient in need thereof, wherein the suboptimal doses are administered at suitable intervals prior to a first round of pre-surgical chemotherapy or radiation, after surgery, and prior to and after a second or repeated course of post-surgical chemotherapy or radiation.

18. A therapeutic composition for inhibiting tumor migration in a mammalian subject comprising one or more of Gamitrinib, an antibody to FAK, an antibody to ULK1, an antibody to mTORC1, an antibody to TRAP1, or a nucleic acid molecule that expresses in a host cell a target selected from AMPK, FIP200 or LKB1

19. A therapeutic composition for inhibiting tumor migration in a mammalian subject comprising one or more of a nucleic acid molecule that bind to a target selected from FAK, ULK1, mTORC1, TRAP1, AMPK, FIP200 or LKB1.
